(19) United States
(12) Patent Application Publication
(10) Pub. No.: US 2010/0144634 A1
(43) Pub. Date: Jun. 10, 2010

(54) Compositions and Methods for Modulation of ADAMTS13 Activity

(76) Inventors: X. Long Zheng, Wallingford, PA (US); Ping Zhang, Brookline, MA (US); Sriram Krishnaswarny, St. Davids, PA (US); Wenjing Cao, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/627,485

(22) Filed: Nov. 30, 2009

Related U.S. Application Data
(63) Continuation-in-part of application No. PCT/US08/65569, filed on Jun. 2, 2008.
(60) Provisional application No. 60/941,245, filed on May 31, 2007.

(51) Int. Cl. A61K 38/16; G01N 33/53

(52) U.S. Cl. 514/12; 435/7.92

(57) ABSTRACT

Compositions and methods are provided for the diagnosis and treatment of thrombotic thrombocytopenic purpura (TTP), stroke and myocardial infarction.

This application is a 35 U.S.C. §365(c) continuation-in-part application of PCT/US08/65569, filed Jun. 2, 2008, which claims priority under 35 U.S.C. §119(e) to U.S. Provisional Application 60/941,245, filed May 31, 2007. The entirety of the foregoing applications is incorporated by reference herein.

Pursuant to 35 U.S.C. §202(c) it is acknowledged that the U.S. Government has certain rights in the invention described, which was made in part with funds from the National Institutes of Health, Grant Numbers HL079027, HL 078726, HL62523, HL47465, and HL081012.

### FIELD OF THE INVENTION

This invention relates to the fields of physiology and hematology. More specifically, the invention provides composition and methods for modulation of ADAMTS13 activity and screening assays to identify agents which augment or inhibit the same. Also provided are compositions and methods for treatment of aberrant thrombus formation such as that observed in TTP and stroke.

### BACKGROUND OF THE INVENTION

Several publications and patent documents are cited throughout the specification in order to describe the state of the art to which this invention pertains. Each of these citations is incorporated herein by reference as though set forth in full.

ADAMTS13 controls the sizes of von Willebrand factor (VWF) multimers by cleaving VWF at the Tyr¹⁶⁰⁵-Met¹⁶⁰⁶ bond at the central A2 domain¹. Deficiency of plasma ADAMTS13 activity, due to either inherited mutations of ADAMTS13 gene²⁻⁹ or acquired autoantibodies against ADAMTS13 protein^{10;11} results in thrombotic thrombocytopenic purpura (TTP).

ADAMTS13 is primarily synthesized in hepatic stellate cells¹²⁻¹⁴, endothelial cells^{15;16} and megakaryocytes or platelets^{17;18}. The plasma ADAMTS13 in healthy individuals ranges from 0.5 mg to 1 mg per liter^{19;20}. ADAMTS13 consists of metalloprotease, disintegrin, first thrombospondin type 1 (TSP-1) repeat, Cys-rich and spacer domains^{2;21}. The C-terminus of ADAMTS13 has additional TSP1 repeats and two CUB domains^{2;21}. Previous studies have shown that the N-terminus of ADAMTS13 are required and sufficient for recognition and cleavage of denatured multimeric VWF²²⁻²⁴ or peptide substrate (GST-VWF73 or FRETS-VWF73)²². More recent studies have demonstrated that the spacer domain of ADAMTS13 binds the exosite (E¹⁶⁶⁰ APDLVLQR¹⁶⁶⁸) near the C-terminus of the VWF-A2 domain²⁵′²⁶. However, the role of the middle and distal C-terminal domains of ADAMTS13 in substrate recognition remains controversial. On the one hand, ADAMTS13 mutant lacking the CUB domains or truncated after the spacer domain cleaved multimeric VWF with similar efficiency as the full-length ADAMTS13 under static and denatured condition^{23;24}; the mutant truncated after the spacer domain, when mixed with ADAMTS13 mutant deleted after the spacer domain, was found to be “hyperactive” in cleaving “string-like” structure, which represents platelets attached to the newly released VWF on endothelial cell surface in a parallel flow chamber-based assay²⁷. These data indicate that the distal portion of ADAMTS13 molecule may be dispensable under static and denatured condition, but may play a role in modulating ADAMTS13-VWF interaction under flow. On the other hand, synthetic peptides or recombinant fragments derived from the CUB domains²⁸ appeared to block the cleavage of the “string-like” structure on endothelial cells, indicating that the TSP1 repeats and CUB domains may directly participate in binding or recognition of VWF under flow. Although the parallel-flow chamber assay may mimic physiological condition, its complexity involving live endothelial cells, histamine stimulation, and platelets makes the quantitation less accurate and kinetic determination of ADAMTS13 and VWF interaction impossible.

### SUMMARY OF THE INVENTION

In accordance with the present invention, a method for analyzing the VWF cleaving action of ADAMTS13 and variants thereof is provided. An exemplary method entails providing VWF and contacting the VWF with intact ADAMTS13 and truncated variants thereof under conditions suitable for enzymatic cleavage of VWF. The amount of VWF cleavage in the presence of full length ADAMTS13 relative to that observed in the presence of said truncated variants is then determined, thereby identifying a minimal ADAMTS13 sequence suitable to effect cleavage of VWF. In a preferred embodiment, the method is performed under flow.

In a further aspect, the method further comprises screening test compounds which modulate ADAMTS13 mediated cleavage of VWF. One compound so identified is Factor VIII which increases ADAMTS13 VWF cleaving activity.

In yet another embodiment of the invention, a method for diagnosing TTP in a patient is provided. A biological sample comprising VWF and ADAMTS13 is obtained from the patient and subjected to vortex induced shear stress. The level of VWF cleavage in the biological sample relative to an identically treated sample from a normal patient is then compared, wherein a reduction in VWF cleavage relative to that observed in said normal patient sample is indicative of TTP.

Finally, methods for alleviating the symptoms of TTP, myocardial infarction and/or stroke in a patient in need thereof comprising administration of an effective amount of ADAMTS13 and FACTOR VIII in a biologically compatible medium are also provided.

### BRIEF DESCRIPTION OF THE DRAWINGS

FIG. 1. Constructs of ADAMTS13 and truncated variants. The full-length ADAMTS13 (FL-A13) and the variants truncated after the 8^{th} TSP-1 repeat (delCUB) and after the spacer domain (MDTCS) were cloned into pcDNA3.1 V5-His TOPO vector. The original signal peptide and propeptide of ADAMTS13 were included. The CUB domains (CUB, C1192-T1427), T2-8 repeats (T2-8, W686-W1076), T5-8 repeats (H884-W1076), the CUB domains plus the TSP1 5-8 repeats (T5-8CUB, H884-A1191) and the CUB domains plus the TSP1 2-8 repeats (T2-8CUB, W686-A1191) were cloned into pSecTag/FRT/V5-His TOPO, in which an IgK secretion peptide and a Flag epitope (-DDDDK-) were engineered at the N-terminus of the CUB, T2-8, T5-8, T5-8CUB and T2-8CUB. All constructs contain V5-His epitopes at their C-termini to facilitate purification and detection.

FIG. 2. Proteolytic cleavage of VWF and VWF73 under flow or static condition by ADAMTS13 and C-terminal truncated variants. A. Rotation-speed dependent cleavage of VWF by ADAMTS13: Native plasma VWF (37.5 μg/ml or 150 nM) was incubated with ADAMTS13 (−60 nM) for 1 min and then vortexed for 3 min at 22° C. at rotations speed of from 0 to ˜3,200 rpm (set at “0-10”). B. Dose-dependent cleavage of VWF by ADAMTS13: VWF (18.75 μg/ml or 75 nM) was vortexed for 3 min without (lane 1 and lane 7) or with various concentrations of rADAMTS13 (lane 2-6) or 2.5 μl of normal human plasma with 30 μg/ml (lane 8) or 60 μg/ml (lane 9) of heparin or TTP patient plasma (lane 10). C. Cleavage of VWF by ADAMTS13 and variants: VWF (18.75 μg/ml or 75 nM) was incubated and vortexed for 3 min without (−) or with (+)-60 nM of FL-A13, delCUB and MDTCS in absence (−) or presence (+) of 10 mM EDTA. D. Cleavage of guanidine-HCl denatured VWF by ADAMTS13 and variants: Denatured VWF (37.5 μg/ml or ˜150 nM) was incubated without (−) or with (+) ˜60 nM of purified FL-A13, delCUB and MDTCS in absence (−) or presence (+) of 10 mM EDTA for 1 h. All the reactions above were quenched by addition of SDS-sample buffer and heated at 100° C. for 5 min. The cleavage product (dimer of 176-kDa) was determined by Western blot with peroxidase-conjugated rabbit anti-VWF IgG, followed by chemiluminescent ECL reagents. The signal was obtained by exposure to X-ray film within 5-30 sec. E. Cleavage of GST-VWF73-H by ADAMTS13 and variants: GST-VWF73-H at various concentrations (0˜200 nM) was incubated with ˜60 nM of FL-A13, delCUB and MDTCS for 10 min at 37° C. The cleavage product (34.4 kDa, arrow heads indicated) was determined by Western blot with rabbit anti-GST IgG and Alexa Fluor680 conjugated anti-rabbit IgG. F. The plot of the fluorescent signal: obtained by Odyssey infrared fluorescent image system against concentrations of GST-VWF73 substrate.

FIG. 3. Kinetic binding interaction between VWF and ADAMTS13 (or variants) under flow. A. Effect of flow rates on binding of VWF to ADAMTS13: Purified VWF (18.75 μg/ml or 50 nM) was injected at various flow rates for 3-5 min over the CM5 surface immobilized with FL-A13 in absence of EDTA. B-D: Binding of VWF to ADAMTS13 and C-terminal truncated variants. Purified VWF at various concentrations (0-250 μg/ml or 0-1,000 nM) was injected over the surfaces immobilized by FL-A13 (B), delCUB (C) and MDTCS (D). After equilibrium was established, the HBS-T buffer was then injected over the surface to allow the dissociation to occur. The representative sensograms in absence of EDTA are shown in A-D. The maximal response units (RUₘₐₓ) at equilibrium (y-axis) were obtained from the sensograms and plotted against various concentrations of VWF injected (x-axis). The entries in E and F are the mean of 2-4 repeats in absence (E) or presence (F) of 10 mM EDTA. The equilibrium dissociation constant, K_{D} was calculated by fitting the data to the binding isotherm using non-linear regression.

FIG. 4. Binding of denatured VWF to ADAMTS13 and C-terminal truncated variants. Purified VWF pre-treated for 2 h with 1.5 M guanidine-HCl at 37° C. was diluted (1:10) with HBS-T buffer with or without EDTA into various concentrations (0-125 μg/ml or 0˜500 nM). The diluted VWF was then injected at 20 μl/min for 3 min over the CM5 chips covalently coupled by FL-A13 (A), delCUB (B) and MDTCS (C). After the equilibrium was established, the HBS-T buffer without VWF was flowed over the surface to allow the dissociation phase to be recorded. The equilibrium constant, K_{D}, was determined similarly as described in FIG. 3. The entries in D represent the means±SD of 6 repeats.

FIG. 5. Binding of ADAMTS13 (or variants) to VWF immobilized on solid surfaces. A. Binding of ADAMTS13 and variants to VWF immobilized on a microtiter plate: FL-A13, delCUB and MDTCS (0-200 nM) were incubated without (control) or with VWF (10 μg/ml, 100 μl/well) immobilized on a microtiter plate for 1 h. The bound ADAMTS13 and variants were determined by mouse anti-V5 IgG, followed by rabbit anti-mouse IgG, peroxidase-conjugated and OPD-H₂O₂. The K_{D} (S) was determined by fitting the data into non-linear regression. B. Binding of ADAMTS13 and variants to immobilized VWF on Affi-gel 10: FL-A13, delCUB, MDTCS and metalloprotease domain (M) (˜50 nM) were incubated at 37° C. for 1 h without (−) or with (+) VWF covalently immobilized onto the Affi-gel 10. After extensive washing with TBS and 20 mM Tris-HCl, pH 7.5, 500 nM NaCl, the bound ADAMTS13 and variants were eluted from the beads with SDS-gel sample buffer and determined by Western blot with anti-V5. The amount of input FL-A13, delCUB, and MDTCS is the same with the signal of only FL-A13 shown in lane 1 (IN).

FIG. 6. Binding of VWF to the C-terminal fragments of ADAMTS13 under flow. Purified VWF in HBS-T (0-500 μg/ml or 0-2,000 nM) was injected at 20 μl/min for 3 min over the CM5 surface covalently coupled to CUB (A), T5-8CUB (B) or T2-8CUB (C). After equilibrium was established, the HBS-T was injected to allow the dissociation phase to be recorded. The K_{D} was determined similarly as described in the Materials and Methods. The entries in D represent the means±SD of four repeats (N=4).

FIG. 7. Inhibition of VWF proteolysis by ADAMTS13 under flow by the C-terminal fragments of ADAMTS13. A. The C-terminal fragments blocks cleavage of VWF by ADAMTS13. Purified VWF (18.75 μg/ml or 50 nM) was incubated 10 mM EDTA (control) or 0-150 nM of CUB, T2-8, T5-8, T5-8CUB and T2-8CUB (lane 2-6) for 60 min. ADAMTS13 (50 nM) was then added into the reaction mixture in presence of 50 mM HEPES buffer containing 0.25% BSA, 5 mM CaCl₂ and 0.25 mM ZnCl₂ (total volume, 20 μl) in a 0.2 ml PCR tube with dome caps. The reaction mixture was subjected to vortexing at a fixed rotation rate of ˜2,500 rpm (set “8”) for 3 min on a mini vortexer. The cleavage of VWF was determined by Western blot with anti-VWF IgG, peroxidase conjugated and ECL reagents (arrowheads indicate the dimers of 176 kDa). B. Quantitation of chemiluminescent signal. The signal on X-ray film within the 30 sec to 1 min was quantified by densitometry using NIH ImageJ software. The relative proteolytic activity of ADAMTS13 (%) after being inhibited by various C-terminal fragments was plotted against the concentrations of C-terminal fragments of ADAMTS13 added into the reaction.

FIG. 8. A schematic diagram illustrating how clemencies in VWF-protease cause TTP.

FIG. 9. Factor VIII enhances the cleavage of rVWF by ADAMTS13 under flow. Shown are western blot and graph illustrating that the addition of recombinant Factor VIII to a reaction mixture containing VWF and ADAMTS13 significantly enhances cleavage of VWF.

FIG. 10. FVIII enhances proteolytic cleavage of multimeric vWF by ADAMTS13 under shear stress. Panel A: Plasma-derived vWF (pvWF) or recombinant vWF (rvWF) (150 nM) was incubated without (−) and with (+) ADAMTS13 (50 nM) in the absence (lane 1) and the presence of the indicated concentrations of FVIII (lanes 2-9). Lane 9 contained 40 nM FVIII plus 20 mM EDTA. The 350K cleavage product was visualized by Western blot analysis following 3 minutes of vortexing. A non-specific, preexisting band in the vWF preparations of unknown origin which also accumulates with proteolysis is denoted by an asterisk. Panel B: Increase in cleavage product detected relative to that observed in the absence of FVIII (Fold Increase) was determined by densitometry. Results represent the mean±standard deviation of 4 independent experiments.

FIG. 11. FVIII preferentially accelerates cleavage of high molecular weight vWF by ADAMTS13 under shear stress. pvWF (150 nM) was incubated with recombinant ADAMTS13 (50 nM) in the absence (−) and presence (+) of 20 nM FVIII and vortexed at 2,500 rpm for the indicated times. Proteolysis was assessed by immunological detection of multimers (Panel A) or the detection of the Mr=350K fragment (Panel B). HMW denotes high molecular weight multimers.

FIG. 12. FVIII has no effect on cleavage of denatured vWF under static conditions. pvWF (150 nM) pretreated with guanidine was incubated for 1 hour at 37° C. with recombinant ADAMTS13 (12.5 nM) in the absence (Panel A, lane 1) and the presence (Panel A, lanes 2-7) of the indicated concentrations of FVIII. Panel A, lane 7 contained 40 nM FVIII plus 20 mM EDTA. Proteolysis was assessed by immunological detection of the 350K fragment. Asterisk indicates the pre-existing band in the vWF preparation. Panel B: Dependence of product formation on the concentration of FVIII was determined by densitometry analysis and is presented as mean±standard deviation of 3 experiments.

FIG. 13. Proteolytic activation alters FVIII effects on vWF cleavage by ADAMTS13 under shear stress. Panel A: SDS-PAGE analysis of purified FVIII (lane 2) and FVIIIa (lane 3) 30 seconds after incubation with thrombin. Protein bands were visualized by staining with SYPRO Ruby fluorescent dye. Lane 1 contains markers with the indicated molecular weights (×10³). HC, LC, A1 and A2 denote heavy chain, light chain, A1 and A2 fragments. Panel B: pvWF (150 nM) was incubated with recombinant ADAMTS13 (50 nM) under constant vortexing for 3 min in the absence (lane 1), in the presence of 20 nM FVIII (lane 2), and at the indicated times following rapid activation of 20 nM FVIII with 20 nM human thrombin and quenched with 30 nM hirudin (lanes 4-6). vWF proteolysis was assessed by immunological detection of the Mr=350K fragment. Panel C: Product formation relative to that observed in the presence of ADAMTSI3 alone was determined by quantitative densitometry. Results are presented as mean±standard deviation of 3 experiments.

FIG. 14. Properties of FVIII derivatives: Panel A: Schematic representation of the domain structure of FVIII and derivatives. The heavy chain composed of AI-A2 domains is linked to a heterogeneously processed B-domain of variable length. The light chain is composed of A3-CI-C2 domains. The three acidic regions are denoted as a1, a2 and a3. FVIII-SQ is secreted as a two-chain molecule in which the heavy chain contains 14 residues of the B domain. FVIII-2RKR is similar to FVIII-SQ except it lacks a3. Panel B: FVII-SQ and FVIII [symbol] 2RKR prior to and after activation by thrombin were analyzed by SDS-P AGE and visualized by staining with Coomassie Blue. HC, LC, A1 and A2 denote the positions of the heavy and light chains, and A1 and A2 domains. Lane 1 contains molecular weight markers with the indicated molecular weights (xlO\ Panel C: Binding of increasing concentrations of FVIII [symbol] SQ or FVIII-2RKR to immobilized vWF detected in an ELISA format.

FIG. 15. FVIII-SQ but not FVIII-2RKR enhances proteolytic cleavage ofvWF by ADAMTS13 under shear stress. Panel A. pvWF (150 nM) was incubated with recombinant ADAMTS13 (50 nM) in the presence of the indicated concentrations of FVIII-SQ or FVIII [symbol] 2RKR for 3 min under vortexing at 2,500 rpm. Proteolysis was assessed by immunological detection of the M:r=350K fragment (Panel A) followed by densitometry analysis of product formed normalized to the product observed in the absence of FVIII derivative (Panel B). Means±standard deviations from 3 experiments are illustrated.

FIG. 16. Cleavage of cell-bound ultralarge (UL) von Willebrand factor (VWF) by recombinant ADAMTS13 and variants. Panel A: Immunofluorescent microscopic images of UL VWF polymers on endothelial cells. The arrowheads indicate the UL VWF strings or bundles. The bar in each figure represents 25 μm. Panel B: Enzyme-linked immunosorbent assay (ELISA) was used to determine the VWF antigen released from endothelial cells in the conditioned medium. This panel shows the VWF antigen levels in the conditioned media of histamine-stimulated HUVECs that were treated without (dashed line, +buffer) or with (solid line, +ADAMTS13) ADAMTS13 (10 nM) for 0, 2, 5, 10, 30 and 60 min (Bi) or for 10 min with various concentrations of ADAMTS13 (0, 1, 2.5, 5, 10, 20 and 40 nM) (Bii) in the absence of flow. Three independent experiments (n=3) were performed for each data point, and the means±standard deviations (SDs) are shown in both subpanels. Panel C: ELISA quantification of VWF antigen released into conditioned media of HUVECs treated with ADAMTS13 and variants. The difference between FLAD13 (or delCUB or MDTCS) and M (or MDT or buffer) was statistically significant, as shown by the P-values. The arrowheads in (Cii) indicate the proteolytic cleavage products (176 kDa and 140 kDa) under reducing conditions. The ## sign indicates the proteolytic products of VWF by other unidentified proteases or non-specific binding of rabbit anti-VWF IgG to unknown proteins in the highly concentrated conditioned media from HUVECs treated with buffer alone, MDT, and M. Panel D: Immunofluorescent microscopic imaging of UL VWF polymers on HUVECs treated with ADAMTS13 variants. The bar in each figure represents 25 μm. Panel E: VWF multimer distribution in the conditioned media treated with or without ADAMTS13 in the absence or presence of flow. The UL VWF multimers (UL) were present in all conditioned media of HUVECs, regardless of whether they were treated with ADAMTS13 in the absence or presence of flow. However, this UL VWF was not present in the NHP. L indicates the lowest molecular weight form of VWF detected in the conditioned medium of HUVECs in culture; this is larger than the bottom two bands observed in the NHP (Ei, lanes 1 and 9; Eii, lane 1).

### DETAILED DESCRIPTION OF THE INVENTION

ADAMTS13 cleaves von Willebrand factor (VWF) between Tyr and Met¹⁶⁰⁶ residues at the central A2 subunit. The amino-terminus of ADAMTS13 protease appears to be sufficient to bind and cleave VWF under static and denatured conditions. However, the role of the carboxyl-terminus of ADAMTS13 in substrate recognition remains controversial. The present study demonstrates that ADAMTS13 cleaves VWF in a rotation speed- and protease concentration-dependent manner on a mini-vortexer. Removal of the CUB domains (delCUB) or truncation after the spacer domain (MDTCS) abolishes its ability to cleave VWF under the same conditions. ADAMTS13 and delCUB (but not MDTCS) bind VWF under flow with dissociation constants (K_{D}) of ˜50 nM and ˜274 nM, respectively. The isolated CUB domains are neither sufficient to bind VWF detectably, nor capable of inhibiting proteolytic cleavage of VWF by ADAMTS13 under flow. Addition of the TSP1 5-8 (T5-8CUB) or TSP1 2-8 repeats (T2-8CUB) to the CUB domains restores the binding affinity toward VWF and the inhibitory effect on cleavage of VWF by ADAMTS13 under flow. These data demonstrate directly and quantitatively that the cooperative activity between the middle carboxyl-terminal TSP 1 repeats and the distal carboxyl-terminal CUB domains may be crucial for recognition and cleavage of VWF under flow.

The factors that modulate proteolytic cleavage of VWF under flow condition have not been described. Factor VIII and VWF circulate in blood as complexes. To determine whether binding of factor VIII augments VWF proteolysis by ADAMTS13, the effect of native factor VIII and B-domain deleted factor on proteolytic cleavage of VWF by ADAMTS13 was determined. Addition of recombinant factor VIII (rFVIII) or B-domain-deleted factor VIII increases the proteolytic cleavage of VWF by ADAMTS13 by at least ≈10-fold, determined by Western blot and other assays. The half maximal effect of rFVIII on proteolytic cleavage of VWF by ADAMTS13 is estimated to be approximately 2.9 nM. In contrast, addition of rFVIII (up to 40 nM) into pre-denatured VWF (with 1.5 M guanidine-HCl) fails to increase the proteolytic cleavage of such VWF by ADAMTS13. The data indicate that the distal carboxyl-terminal domains of ADAMTS13 appear to be crucial for recognition and cleavage of VWF under flow and coagulation factor VIII binds VWF and may serve as a cofactor to regulate ADAMTS13 proteolytic function under flow shear stress or in vivo.

Also in accordance with the present invention a simple flow-based assay has been developed to determine ADAMTS13 activity. This assay is based on vortex-induced mechanic shear stress that unfolds the globular VWF molecule and allows ADAMTS13 enzyme to access the cleavage bond (Tyr-Met). By simple vortexing at room temperature for 2-5 minutes, the proteolysis of VWF by ADAMTS13 is significantly enhanced. This enhancement of VWF proteolysis is vortex-speed and ADAMTS13 concentration dependent. The cleavage of VWF can be detected in minutes rather than in hours and days as in previously described assays. No denaturing reagents are needed. The assay is simple and reproducible for measuring ADAMTS13 activity under flow. The cleavage of VWF by ADAMTS13 is specific and can be completely blocked by addition of 10 mM EDTA and by TTP patient IgG. No cleavage was detected in TTP patient plasma that is known to have autoantibodies against ADAMTS13. Therefore, this simple vortex-induced flow assay may be used to advantage to study the biological function of ADAMTS13 under flow or modified for clinical use for diagnosis of TTP. The assay is particularly advantageous for analysis of patients exhibiting normal ADAMTS13 activity as determined in static and denatured assays. Also provided is an automatic flow device that vortexes multiple samples at the same time for assessing cleavage of VWF under different conditions. Cleavage could be monitored for example, using alterations in light scattering properties or intrinsic fluorescent changes.

Another aspect of the invention relates to the treatment of stroke and other blood coagulation disorders. Data have shown that low ADAMTS13 activity is a risk factor for myocardial infaraction and ischemic stroke. Indeed, recombinant ADAMTS13 is being tested in a phase I clinical trial for these disorders in addition to assessing efficacy for the treatment of TTP. The in vivo data demonstrate that mice lacking FVIII exhibit compromised vWF degradation upon hydrodynemic challenge, which gives rise to prothrombotic events. In humans, VWF antigen and multimers are increased in patients with severe hemophilia A (lacking FVIII), indicating that FVIII is a physiological cofactor accelerating vWF proteolysis by ADAMTS13 enzyme. The discovery of this cofactor activity of FVIII provides the basis for a therapeutic regimen that is more effective for anti-thrombotic applications. Considering the number of patients with MI and stroke, such regimes provide an advance in the art of treating these conditions.

Thus, ADAMTS13/FVIII administered in combination or as polypeptide complexes may be used for a variety of purposes in accordance with the present invention. In a preferred embodiment of the present invention, ADAMTS13/FVIII polypeptides or complexes may be administered to a patient via infusion in a biologically compatible carrier. The polypeptides or complexes thereof of the invention may optionally be encapsulated in to liposomes or other phospholipids to increase stability of the molecule. The polypeptides or complexes there of may be administered alone or in combination with other agents known to modulate thrombotic events. An appropriate composition in which to deliver ADAMTS13/FVIII polypeptides or complexes thereof may be determined by a medical practitioner upon consideration of a variety of physiological variables, including, but not limited to, the patient's condition and hemodynamic state. A variety of compositions well suited for different applications and routes of administration are well known in the art and described hereinbelow.

The preparation containing the purified polypeptides or complexes contains a physiologically acceptable matrix and is preferably formulated as a pharmaceutical preparation. The preparation can be formulated using substantially known prior art methods, it can be mixed with a buffer containing salts, such as NaCl, CaCl₂, and amino acids, such as glycine and/or lysine, and in a pH range from 6 to 8. Until needed, the purified preparation containing the polypeptides or polypeptide complex can be stored in the form of a finished solution or in lyophilized or deep-frozen form. Preferably the preparation is stored in lyophilized form and is dissolved into a visually clear solution using an appropriate reconstitution solution.

Alternatively, the preparation according to the present invention can also be made available as a liquid preparation or as a liquid that is deep-frozen.

The preparation according to the present invention is especially stable, i.e., it can be allowed to stand in dissolved form for a prolonged time prior to application.

The preparation according to the present invention can be made available as a pharmaceutical preparation with anti thrombotic activity in the form of a one-component preparation or in combination with other factors in the form of a multi-component preparation.

Prior to processing the purified proteins into a pharmaceutical preparation, the purified proteins are subjected to the conventional quality controls and fashioned into a therapeutic form of presentation. In particular, during the recombinant manufacture, the purified preparation is tested for the absence of cellular nucleic acids as well as nucleic acids that are derived from the expression vector, preferably using a method, such as is described in EP 0 714 987.

Another feature of this invention relates to making available a preparation which contains ADAMTS13 and FVIII with high stability and structural integrity and which, in particular, is free from inactive intermediates and autoproteolytic degradation products.

The pharmaceutical preparation may contain dosages of between 10-1000 μg/kg, more preferably between about 10-250 μg/kg and most preferably between 10 and 75 μg/kg, with 40 μg/kg of the polypeptides being particularly preferred. Patients may be treated immediately upon presentation at the clinic with a coagulation disorder or thrombotic disorder. Alternatively, patients may receive a bolus infusion every one to three hours, or if sufficient improvement is observed, a once daily infusion of the polypeptides described herein.

The following examples are provided to illustrate certain embodiments of the invention. They are not intended to limit the invention in any way.

### Example I

### The Cooperative Activity Between the Carboxyl-Terminal TSP-1 Repeats and the CUB Domains of ADAMTS13 is Crucial for Recognition of Von Willebrand Factor Under Flow

In the present study, a simple flow assay was developed based on mechanical-induced shear stress on a mini vortexer or a laminar flow in a BIAcore system to determine the role of the C-terminal ADAMTS13 in recognition and cleavage of multimeric VWF. The data demonstrate directly and quantitatively the cooperative activity between the middle C-terminal TSP1 repeats and the distal C-terminal CUB domains of ADAMTS13 may be crucial for productive binding and cleavage of VWF under flow.

The following materials and methods are provided to facilitate the practice of the present invention.

### Constructs:

The plasmids containing full-length ADAMTS13 (FL-A 13) and variant truncated after the 8^{th} TSP1 repeat (delCUB) or after the spacer domain (MDTCS) and the metalloprotease domain (M) were described previously^{22;24;29}. The cDNA fragments encoding the CUB domains (CUB), TSP1 2-8 (T2-8), TSP1 5-8 (T5-8), TSP1 5-8 repeats plus CUB domains (T5-8CUB) and TSP1 2-8 plus CUB domains (T2-8CUB) were amplified by PCR using pcDNA3.1-FL-A13 as a template and cloned into pSecTag/FRT/V5-HisTOPO (Invitrogen, Carlsbad, Calif.) according to manufacturer's recommendation. The constructs CUB, T2-8, T5-8, T5-8CUB and T2-8CUB were tagged at their N-termini with a linker sequence and a flag (underlined) epitope (AAQPARRARRTKLA-LDTKDDDDKHVWTPVA-) and C-termini with V5-His epitope. The plasmids were sequenced to confirm the accuracy.

### Cell Culture and Transfection:

The human embryonic kidney cells (HEK-293) grown in Dulbecco's modified Eagle's medium (DMEM) (Invitrogen, Carlsbad, Calif.) containing 10% of FetalPlex (Gemini BioProducts, West Sacramento, Calif.) were transfected with mixture of LipofectAMINE2000 and plasmids (3:1, vol: weight) in serum-free Opti-MEM. Constructs in pSecTag/FRT/V5-His vector were co-transfected with pcDNA3.1 vector (Invitrogen) to obtain the neo gene for stable selection. After 72 hours of transfection, the stable clones were selected by treating the cells with 0.5 mg/ml of geneticin (G418) (Invitrogen, Carlsbad, Calif.) and identified by Western blotting with anti-V5 IgG (Invitrogen, Carlsbad, Calif.) as described previously^{22;24}.

### Preparation of Recombinant Proteins:

Stably transfected HEK-293 cells expressing ADAMTS13 and variants were cultured on 10-layer cell factories (Fisher Scientific) in Opti-MEM (Invitrogen, Carlsbad, Calif.) or serum-free DMEM supplemented with 5 mg/ml of insulin transferring selenium (ITS) (Roche Applied Science, Indianapolis, Ind.) supplement at 80% confluency. The conditioned medium (˜2 liters) was collected every 24 to 48 hours and the cell debris was removed by centrifugation at 3,000 rpm for 10 min and filtration through coarse filter paper (Fisher Scientific). After addition of 5 mM benzamidine and 1 mM phenylmethylsulfonyl fluoride (PMSF) (Sigma, St. Louis, Mo.), the conditioned medium was frozen and stored at −80° C. until use.

The conditioned medium was thawed at room temperature and diluted (1:3) with distilled water. The pH was adjusted to 8.0 by adding 2 M Tris-HCl, pH 8.0. The diluted conditioned medium was loaded onto Q-fast flow ion exchange column (125 ml) at 4° C. overnight. After being washed with 20 mM Tris-HCl, pH 8.0, the protein was eluted with 5-10 column volumes of 1 M NaCl in 20 mM Tris-HCl, pH 8.0. The fractions containing proteins were pooled and then loaded onto 10-80 ml Ni-NTA affinity column (Invitrogen, Carlsbad, Calif.). After being washed with 20 mM Tris-HCl, pH 8.0, 400 mM NaCl in presence of 10 mM imidazole, the bound proteins were eluted with 60 mM imidazole in 20 mM Tris-HCl, pH 8.0 and 400 mM NaCl. The fractions (4 ml each) were collected and the peak fractions containing recombinant proteins of interest were pooled and concentrated with Centri-Prep30 (Millipore, Billerica, Mass.). The proteins were further separated by Superose 6 10/300 GL gel filtration chromatography (GE Biosciences, Piscataway, N.J.) at 0.5 ml/min with 20 mM Tris-HCl, 150 mM NaCl, pH 7.5 as described previously²². The SDS-polyacrylamide gel-electrophoresis and Coomassie blue staining determined the molecular weight and purity of purified proteins. The amount of the purified proteins was determined by absorbance at 280 nm (corrected with light scattering at 340 nm) with absorbance coefficients of 0.68 (FL-A13), 0.71 (delCUB), 0.91 (MDTCS), 0.63 (CUB), 0.62 (T2-8), 0.81 (T5-8), 0.68 (T5-8CUB), and 0.60 (T2-8CUB) mg ml⁻¹ cm^{-1 30;31}. The amount of specific ADAMTS13 antigen was also verified by Western blot with anti-V5 using Positope™ (Invitrogen) as a standard.

### Cleavage of VWF Under Flow and Static Condition:

Purified plasma-derived VWF (37.5 μg/ml or 150 nM, final concentration)^{22;24} was incubated with ADAMTS13 and variants at concentrations indicated in each figure and figure legend in 50 mM HEPES buffer containing 0.25% BSA, 5 mM CaCl₂ and 0.25 mM ZnCl₂ (total volume, 20 μl) in a 0.2 ml thin-walled PCR tube with dome caps (Fisher Scientific, Hampton, N.H.) for 1 min. Here the molar concentration of VWF was calculated using a molecular weight of 250 kDa for each VWF polypeptide as described previously³⁰. The reaction mixture was subjected to vortexing at a fixed rotation rate of ˜2,500 rpm (set “8”) or various rotation speeds between 0 and ˜3,200 rpm for 3 min on a mini vortexer (Fisher Scientific, Hampton, N.H.)³².

Alternatively, purified plasma-derived VWF was incubated with 1.5 M guanidine-HCl at 37° C. for 2 hours^{1;10}. The denatured VWF was diluted 1:10 with 50 mM HEPES buffer containing 0.25% BSA, 5 mM CaCl₂ and 0.25 mM ZnCl₂³⁰. Denatured VWF (37.5 μg/ml or 150 nM) was incubated with ˜60 nM of ADAMTS13 (or variants) at 37° C. for 1 hour. The reaction was quenched by heating the samples at 100° C. for 10 min after addition of sample buffer (0.625 mM Tris-HCl, pH 6,8, 10% Glycerol, 2% SDS and 0.01% bromphenol blue). The cleavage products were detected by Western blot with peroxidase-conjugated anti-VWF IgG (p0226, DAKO) (1:3,000) in 1% casein (Sigma, St. Louis, Mo.) or anti-VWF IgG (p082, DAKO) followed by peroxidase-conjugated anti-rabbit IgG (1:5,000), followed by SuperSignal Chemiluminescent reagents (Pierce, Rockford, Ill.).

### Cleavage of GST-VWF73-H by ADAMTS13 and Variants:

The proteolytic cleavage of GST-VWF73 was determined by Western blotting with rabbit anti-GST IgG (Molecular Probes) as described²², followed by Alexa Fluor680 conjugated anti-rabbit IgG (Molecular Probe) (1:12,500). The bound fluorescent antibody was quantified by Odyssey infrared fluorescent image system (LI-COR Bioscience, Lincoln, Nebr.).

### Binding of VWF to ADAMTS13 and Variants Under Flow:

In contrast to a mini vortexer that generates turbulent flow, a BIAcore system produces laminar flow. The shear rate at the inner surface of the injection tube (with diameter of 0.2 mm) can be calculated with a simple equation:

Shear rate≈1.27f/πR³ (Equation 1)

where f is injection flow rate (μl/min) and R is the diameter of the tube (mm). In the micro fluidic cells, the shear rate can also be calculated:

Shear rate≈f/10wh² (Equation 2)

where f is also the injection flow rate (μl/min), w is the side length (mm) and h is the height (mm) of the micro fluidic cell. In BIAcore2000 (BIAcore, Uppsala, Sweden), the dimension of the fluidic cell is 2.4 mm in length, 0.5 mm in width and 0.05 mm in height with a total volume of 60 nL. Accordingly, at injection rate of 1 μl per min, about 50 s⁻¹ shear rate in the inner surface of tube and 80 s⁻¹ shear rate in the micro fluidic cells can be generated. Therefore, the BIAcore system provides us with a unique opportunity to accurately and quantitatively determine the interaction between VWF and ADAMTS13 (or variants) at the single molecule level in real time under flow shear stress.

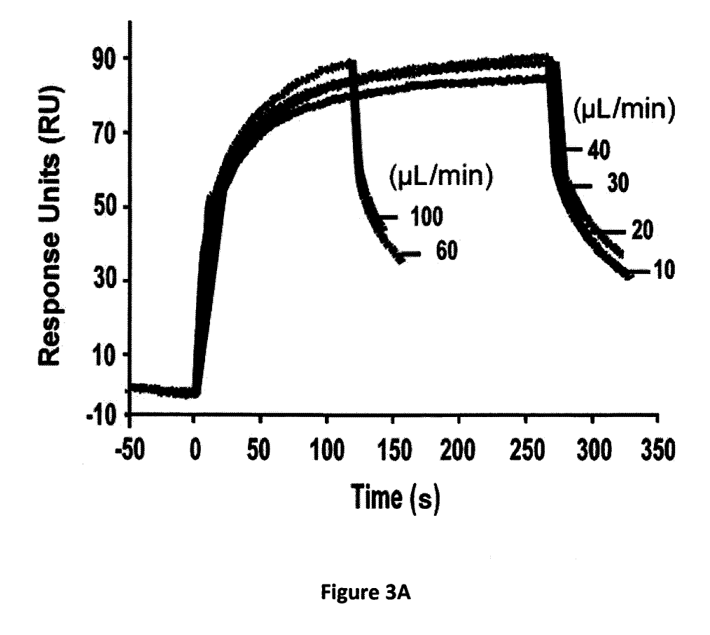

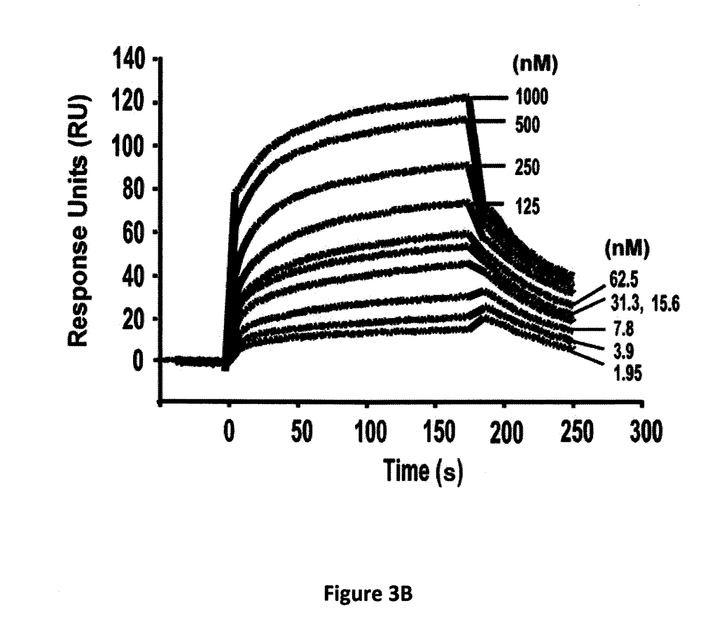

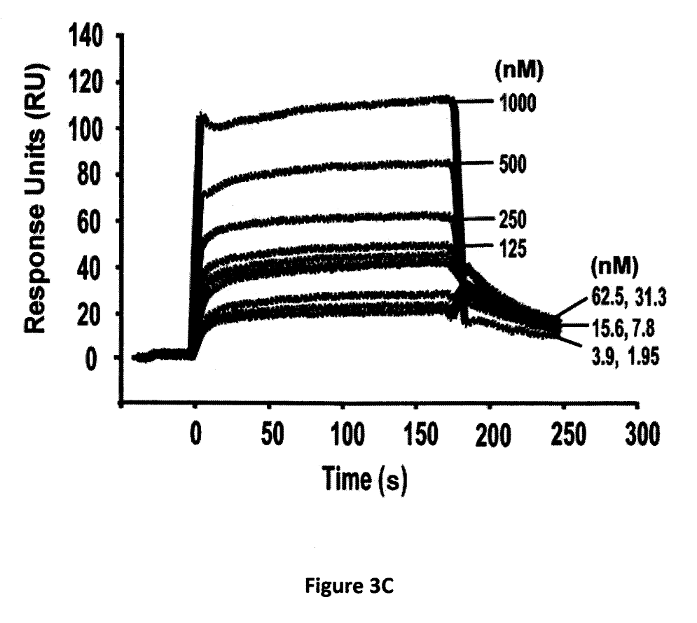

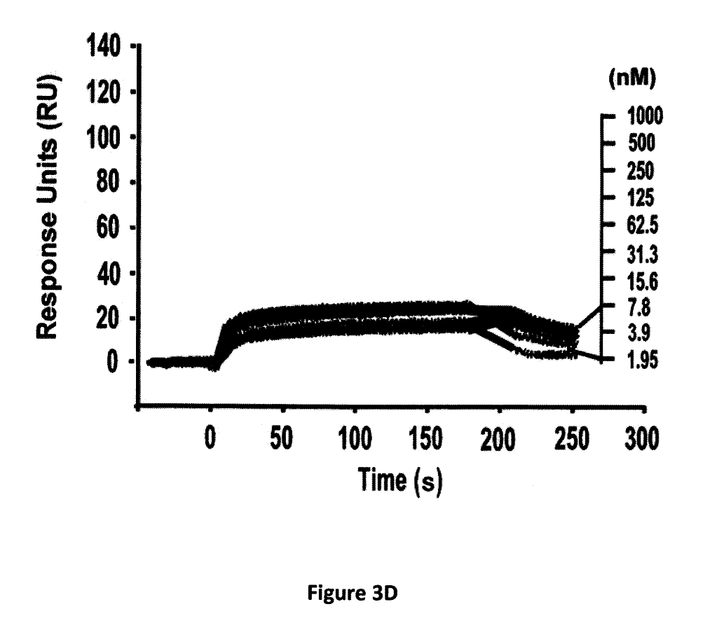

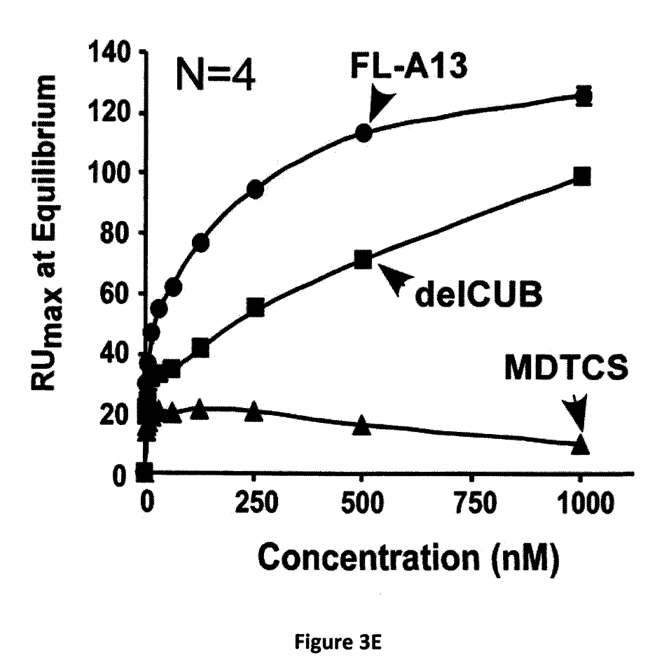

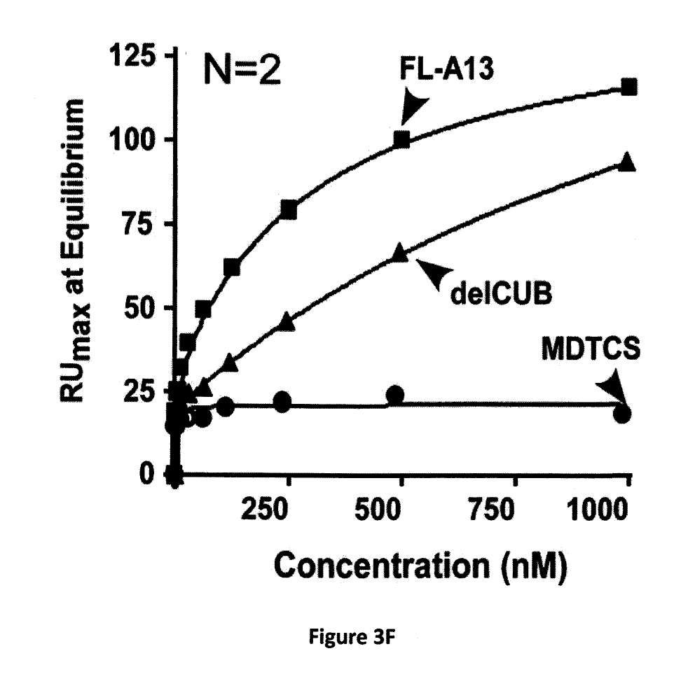

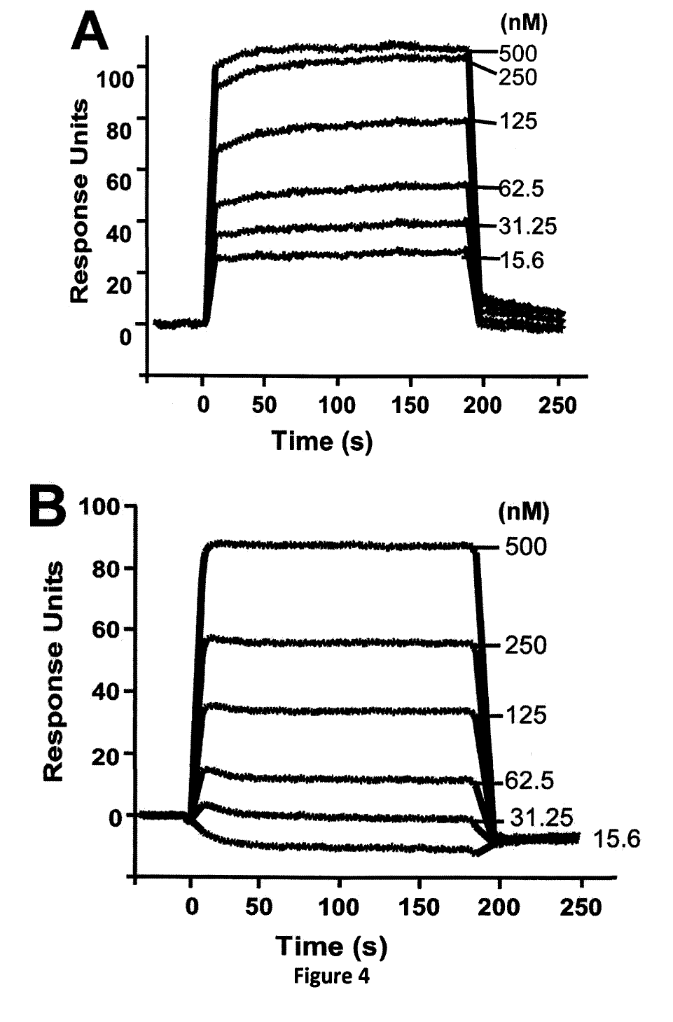

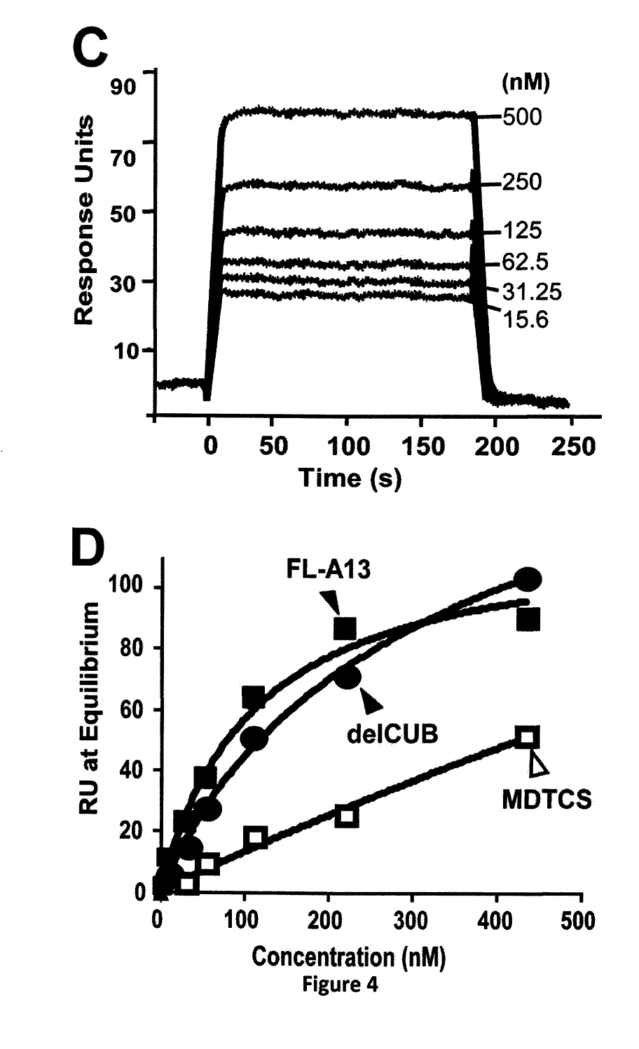

Briefly, the surface of a carboxymethylated dextran (CM5) chip was activated by injection of 35 μl mixture (1:1, vol:vol) of 0.4 M 1-ethyl-3-(3-dimethylaminopropyl) carbodiimide and 0.1 M N-hydroxysuccinimide according to manufacturer's instruction (BIAcore, Uppsala, Sweden). Approximately 2,000 to 8,000 response units (RU) (2˜10 ng/mm²) of purified recombinant proteins were covalently attached onto the activated CM5 chip surface. The control surface was activated similarly, but blocked by same amount of BSA (Sigma, St. Louis, Mo.). The reactive groups on the dextran surface were blocked by injection of 35 μl of 1 M ethanolamine (pH 8.5) at flow rate of 5 μl per min for 7 min. Then, purified plasma VWF at various concentrations (0-250 μg/ml or 0-1,000 nM as in FIG. 3; 0-125 μg/ml or 0-500 nM as in FIG. 4) in 10 mM HEPES, 150 mM NaCl, pH 7.5 containing 0.005% Tween 20 and 2 mg/ml BSA (HBS-T) were injected and passed over the surface at injection rate 10 to 100 μl/min or 20 μl/min for 3-5 min. The HBS-T replaced the protein solution and continued to flow for approximately 4 min; further washing with HBS-T for 20-30 min regenerated the surfaces prior to the next injection. The dissociation constants, K_{D} (S) at the equilibrium were determined by fitting the data from the binding isotherm using a non-linear regression curve on the PRISM4 software (GraphPad Software, Inc., San Diego, Calif.).

### Binding of ADAMTS13 or Variants to VWF Immobilized on Solid Surfaces:

The binding of ADAMTS13 and variants to immobilized VWF on a microtiter plate was performed as described previously²⁹. The specific binding was obtained after subtraction of absorbance in the control wells without VWF ligand. The kinetic parameters were determined by fitting the data into non-linear regression.

The binding of ADAMTS13 to immobilized Affi-gel 10 was also described previously²². Briefly, purified VWF (5 mg) was covalently coupled onto 2 ml of activated Affi-gel-10 (Bio-Rad, Hercules, Calif.) in HEPES buffer, pH 7.5 at 4° C. for 5 hours. The residual reactive groups on the Affi-gel-10 beads were blocked with 0.1 M glycine ethyl ester (Sigma, St. Louis, Mo.), pH 6.5 and 2.5% BSA fraction V (Sigma, St. Louis, Mo.) for 2 hours. The VWF-coupled Affi-gel was stored at 4° C. in 5 mM Tris-HCl, pH 8.0 containing 0.02% sodium azide until use. Ten μl of VWF-Affi-gel (2.5 μg VWF per μl gel) or control Affi-gel that was not coupled with VWF was incubated with approximately 200 nM of FL-A13 (or variants) in 20 mM HEPES, pH 7.5, 150 mM NaCl in presence of 0.25% BSA at 25° C. for 30 min. The beads were washed three times with 10 volumes of 20 mM HEPES, pH 7.5, 150 mM NaCl, and once with 500 mM NaCl. The bound FL-A13 and variants were eluted from the beads by boiling them at 100° C. for 10 min and detected by Western blotting with anti-V5 IgG as described previously^{22;24;29}.

The C-Terminal Fragments of ADAMTS13 Block Cleavage of VWF by ADAMTS13 under Flow:

Purified plasma VWF (37.5 μg/ml or 150 nM) was incubated in absence or presence of 0-150 nM of recombinant CUB, T2-8, T5-8, T5-8CUB and T2-8CUB in 50 mM HEPES buffer containing 5 mM CaCl₂, 0.25 mM ZnCl₂ and 2 mg/ml BSA for 60 min. Then ADAMTS13 (−50 nM) was added and the mixture was subjected to vortexing at 2,500 rpm (set “8”) for 3 min at 22° C. The reaction was quenched as described above by heating the sample in 1×SDS-sample buffer at 100° C. for 5 min. Western blotting as described above determined the cleavage of VWF.

### Results

### Purification of Recombinant ADAMTS13 and Variants:

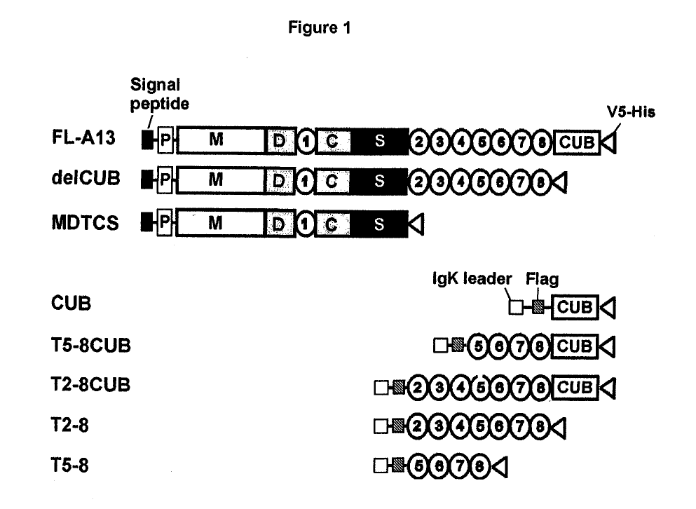

To determine the kinetic interactions between VWF and ADAMTS13 or variants in a purified system, full-length ADAMTS13 and variants or C-terminal fragments were expressed and purified. The domain composition of each construct is listed in FIG. 1. The proteins were purified to homogeneity by three sequential column chromatographies: Q-fast flow ion exchange, Ni-NTA affinity column and Superose 6 gel filtration as described previously²². Typically, approximately 0.2-1.0 mg with ˜90-95% purity of recombinant proteins were obtained from 2 to 10 liters of conditioned medium. The molecular weights of FL-A13, delCUB and MDTCS are estimated to be ˜195 kDa, ˜150 kDa and ˜95 kDa, respectively on SDS-PAGE under denatured and reduced condition. The molecular weights of the constructs CUB, T2-8, T5-8, T5-8CUB and T2-8CUB, however, are ˜50 kDa, ˜100 kDa, ˜52 kDa, ˜95 kDa and ˜116 kDa, respectively.

### Cleavage of VWF by ADAMTS13 and Variants Under Flow:

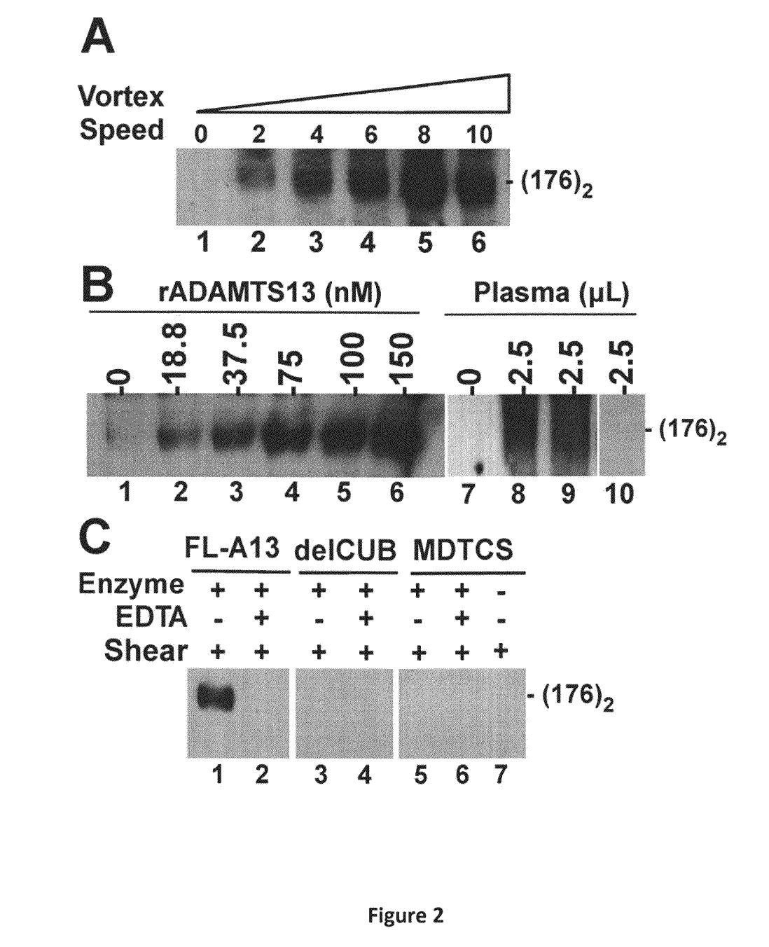

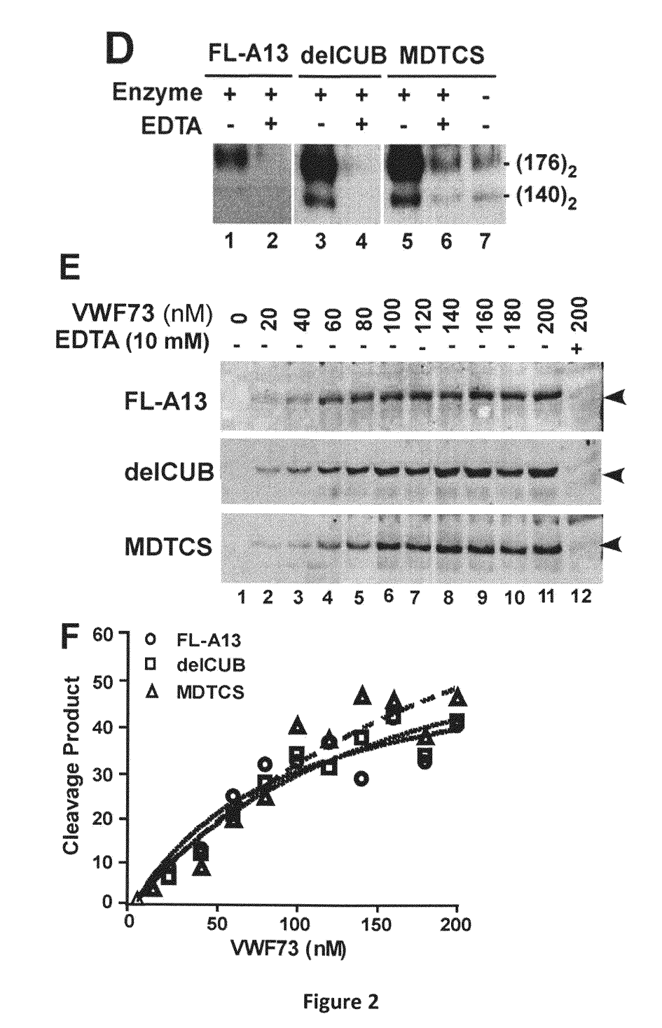

To determine whether the C-terminal domains of ADAMTS13 are required for cleavage of VWF under flow, a simple flow-based assay was developed using a mini vortexer as described elsewhere³². Unique to vortex rotation, turbulent flow that mimics the flow condition in the branching of the vessels or downstream of partially occluded vessels is generated^{33;34}. When vortexing at rotation rates between 640˜3,200 rpm (set “2-8”), VWF was readily cleaved within 3 min by full-length ADAMTS13 in a rotation rate dependent manner; the cleavage product (a dimer of 176-kDa) reached the plateau at rotation rate of ˜2,500 rpm (with estimated shear rate>12,000 s⁻¹)^{33;34} (FIG. 2A); the cleavage of VWF was also ADAMTS13 concentration-dependent at a fixed rotation rate of ˜2,500 (FIG. 2B); even 2.5 μl of normal human plasma was sufficient to cleave VWF in presence of 30-60 μg/ml of heparin under this condition (FIG. 2B). Addition of more heparin (500 μg/ml) and barium chloride (10 mM) increased VWF-cleavage product by plasma ADAMTS13¹. The specificity was confirmed by lack of VWF cleavage product after addition of 10 mM EDTA into the reaction or omitting of ADAMTS13 enzyme or using of TTP-patient plasma (FIG. 2).

Strikingly, a removal of the CUB domains (delCUB) or truncation after the spacer domain (MDTCS) abolished ADAMTS13's ability to cleave VWF under the flow condition at rotation rate of ˜2,500 rpm (FIG. 2C). The same amount of the C-terminal truncated ADAMTS13 was able to cleave guanidine-HCl denatured VWF even more efficiently than full-length ADAMTS13 (FIG. 2D). The constructs FL-A13, delCUB and MDTCS all cleaved GST-VWF73-H (FIGS. 2E and 2F) or FRETS-VWF73 substrate with similar efficacy. The data indicate that the CUB domains of ADAMTS13 are required for cleavage of VWF under turbulent flow.

### Binding of VWF to ADAMTS13 (or Variants) Under Flow:

To determine the binding interaction between VWF and ADAMTS13 (or variants) under laminar flow, the BIAcore technology based on measurement of surface plasmon resonance was employed. Full-length ADAMTS13 or C-terminal truncated variants were attached covalently onto the CM5 surface to avoid VWF activation induced by amine coupling. Purified plasma VWF was then passed in the binding buffer at various concentrations (0˜1,000 nM) over the ADAMTS13 immobilized surfaces. Because plasma VWF multimers vary in sizes and are sensitive to shear stress, injection flow rate may affect the molecule diffusion rate and conformation. To determine diffusion effect or effect of flow rate on VWF-ADAMTS13 binding, a fixed concentration of plasma VWF (12.5 μg/ml or 50 nM) was injected over the surface immobilized by full-length ADAMTS13 at various flow rates (10-100 μl/min) (estimated shear rates between ˜250 s⁻¹ and ˜5,000 s⁻¹). It was found that at various flow rates VWF was able to bind ADAMTS13 with similar association and dissociation kinetics (FIG. 3A). These data indicate that VWF binds ADAMTS13 in high affinity at various flow shear rates. The data also indicate that the VWF-ADAMTS13 binding is not diffusion limited.

Multimeric plasma-derived VWF varies in length and exhibited very fast-association (on) and fast-dissociation (off) rates; the kₒₙ and k_{off} could not be accurately determined. Fitting the data directly using BIAcore evaluation software, although it is relatively easy, may overestimate the binding affinity between VWF and ADAMTS13 due to the heterogeneity of VWF molecules. Therefore, only are the equilibrium dissociation constants, K_{D} (S) reported here. Under the laminar flow, VWF bound full-length ADAMTS13 in a dose- and time-dependent manner (FIG. 3B), with a K_{D} of 50±9.0 nM. A removal of the CUB domains (delCUB) reduced its affinity by 5-fold (K_{D}=274±92 nM) (Mean±SEM) (Table 1 and FIG. 3C). Further removal of the TSP1 2-8 repeats (MDTCS) abolished its affinity toward flowing VWF (FIGS. 3D and 3E). The binding affinity was independent of divalent cations, because addition of 10 mM EDTA into the binding buffer did not affect the binding kinetics or K_{D} values (FIG. 3F). These data demonstrate quantitatively that the distal C-terminal TSP1 repeats and CUB domains may be required for recognition of VWF under flow.

TABLE 1
Kinetic determination of VWF binding to ADAMTS13 (or
variants) by SPR

| | VWF | VWF** |
|---|---|---|
| | K_{D} (× 10⁻⁹M) | K_{D} (×10⁻⁹M) |
| FL-A13 | 50 ± 9 (n = 6) | 83 ± 17 (n = 6) |
| | 77 ± 26 (n = 4) | |
| delCUB | 274 ± 92 (n = 6) | 242 ± 73 (n = 6) |
| | 468 ± 131 (n = 4) | |
| MDTC | No binding | 337 ± 186 (n = 6) |
| | No binding | |

VWF—von Willebrand Factor
VWF** - the VWF substrate was denatured at 37° C. for two hours with 1.5 M guanidine HCl prior to binding experiments
K_{D} the dissociation constant
FL-A13 full length ADAMTS13
delCUB the ADAMTS13 variant truncated after the 8^{th} TSP1 repeat;
MDTCS the variant truncated after the spacer domain;
N = number of repeats performed
The entries are the means ± standard error. The numbers in italics represent data obtained from experiments performed in the presence of 10 mM EDTA.

### Binding of Denatured VWF to ADAMTS13 (or Variants) Under Flow:

It has been shown that addition of 1.5 M guanidine-HCl^{1;10} or 1.5 M urea¹ significantly accelerates VWF proteolysis by ADAMTS13. To determine whether pre-denatured VWF increases its interaction with ADAMTS13 (or variants) under flow, the denatured plasma VWF at various concentrations (0-500 nM) was passed over full-length ADAMTS13 and variants surface. Pre-denatured VWF was able to bind the short construct MDTCS with an increased affinity (K_{D} of 337±186 nM) (Mean±SEM) (N=6) (FIGS. 4C and 4D and Table 1), but the affinity between the denatured VWF and FL-A13 (or delCUB) was not significantly altered with the K_{D} (S) of 83±17 nM and 242±73 nM (Mean±SEM), respectively (Table 1). These data indicate that additional cryptic binding sites that are potentially recognized by the N-terminal domains of ADAMTS13 may be exposed upon pre-denaturization of VWF plus flow shear stress.

### Binding of ADAMTS13 (or Variants) to Immobilized VWF on Solid Surfaces:

VWF can be activated by adsorption onto the solid surfaces²⁹. To validate the specificity of VWF-ADAMTS13 interaction seen in the BIAcore system and to be sure that the purified ADAMTS13 and variants behave as expected in recognition of VWF under a static condition, the binding on a microtiter plate was determined. Consistent with the data reported by Majerus et al²⁹, the recombinant FL-A13, delCUB and MDTCS bound immobilized VWF with K_{D}(S) of 50±6 nM, 70±23 nM and 56±30 nM, respectively (FIG. 5A). The binding interaction was not disrupted by 0.5 M sodium chloride (FIG. 5B), confirming the high affinity binding. The metalloprotease domain alone did not bind immobilized VWF on microtiter palate²⁹ or on VWF-Affi-gel 10 detectably (FIG. 5B), confirming that the recombinant ADAMTS13 and variants are functional and the N-terminal domains of the ADAMTS13 may be sufficient to mediate ADAMTS13 interaction with VWF immobilized/activated on solid surfaces.

### Direct Binding Interaction Between VWF and the C-Terminal Fragments of ADAMTS13 Under Flow:

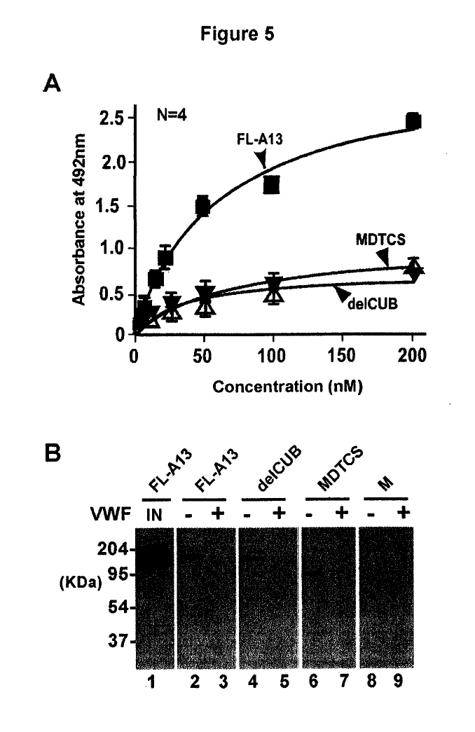

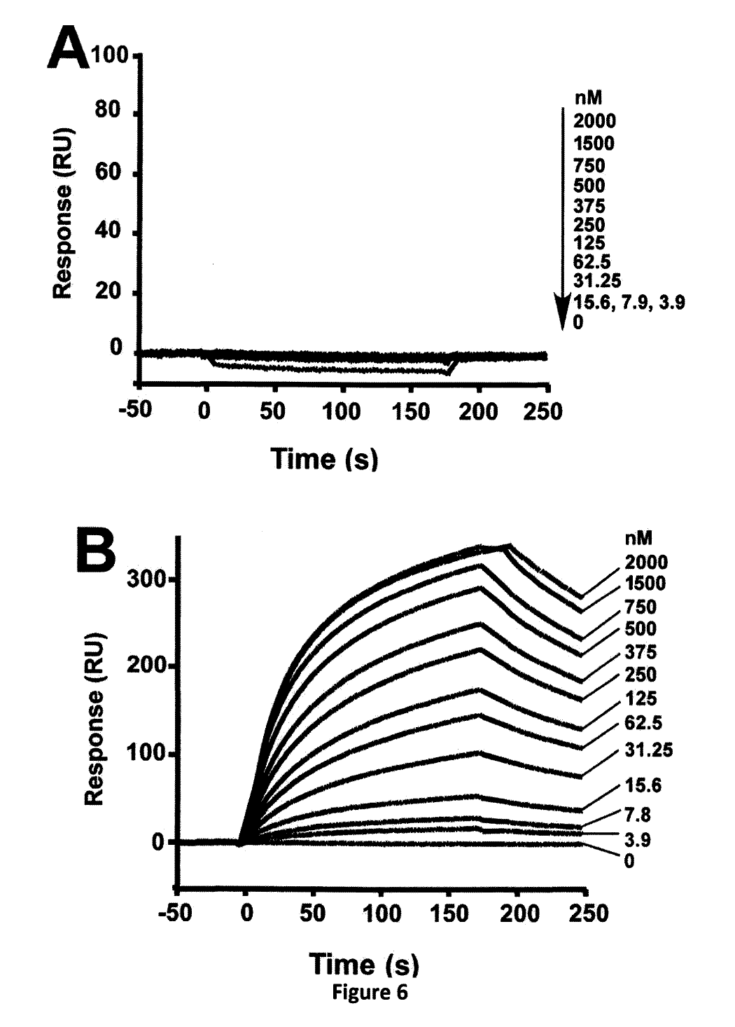

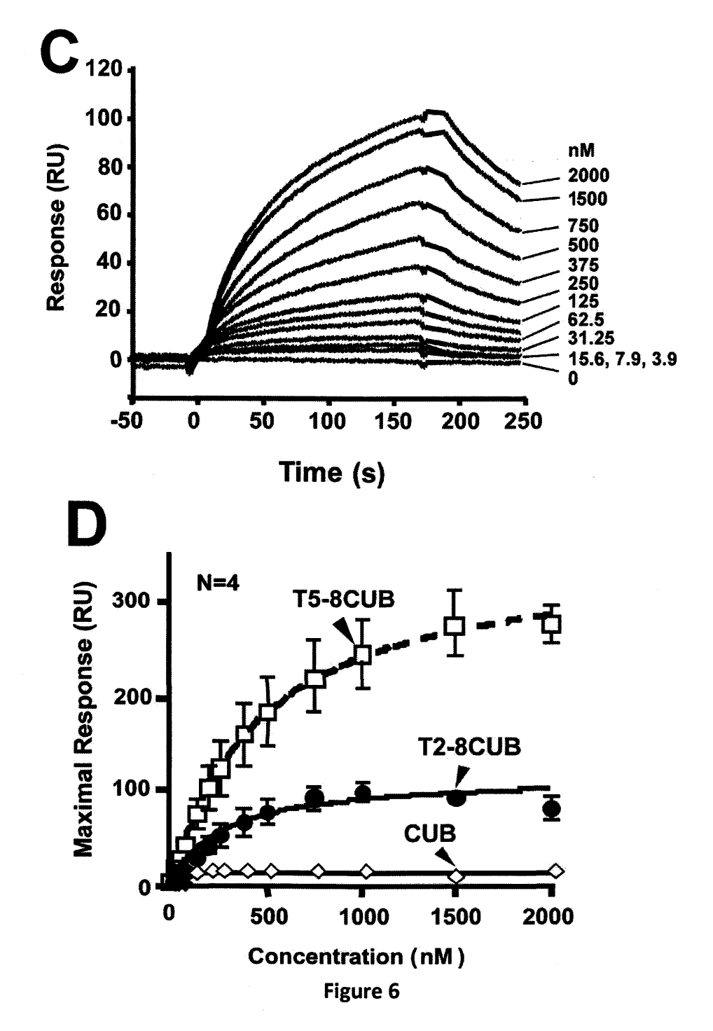

To further determine whether the isolated C-terminal fragments of ADAMTS13 are sufficient to interact with VWF under flow, plasma VWF was injected at various concentrations (0˜1000 nM) and passed it over the surfaces that were covalently attached by nothing, CUB, T5-8CUB and T2-8CUB. Surprisingly, VWF did not bind the isolated CUB domains detectably, but bound the constructs T5-8CUB and T2-8CUB with the K_{D} values of 212±50 nM and 140±36 (means±SEM), respectively (FIG. 6), indicating that the cooperative activity between the distal TSP1 repeats and the CUB domains may be required for productive binding VWF under flow.

### The C-Terminal Fragments of ADAMTS13 Inhibit Cleavage of VWF by ADAMTS13 Under Flow:

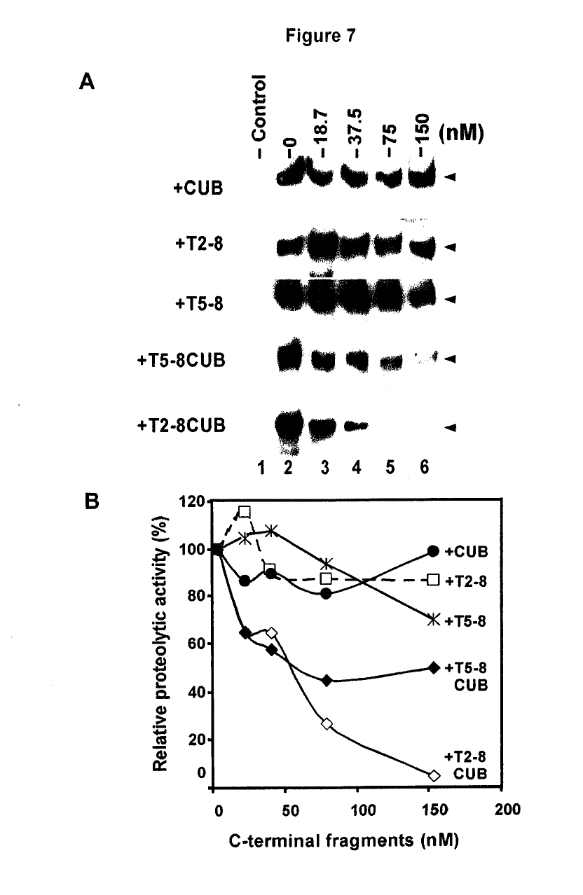

A five-fold reduction in affinity after removal of the CUB domains indicates these domains play a role in recognition of VWF under flow (FIG. 3 and Table 1). However, the immobilized CUB domains alone failed to bind the flowing VWF detectably (FIG. 6A). The discrepancy may be caused by partial deletion of the binding site within distal TSP1 repeats or junction, which cooperates with those in the CUB domains for binding VWF; it may be also caused by the unfavorable orientation of the isolated CUB fragment on the sensor surface. To resolve this discrepancy, a functional inhibition assay on a mini-vortexer was performed. Clearly, when added to the reaction, the CUB domains, TSP1 2-8 or TSP1 5-8 fragment did not significantly inhibit cleavage of VWF by full-length ADAMTS13 dose-dependently (FIG. 7). However, the T5-8CUB and T2-8CUB blocked cleavage of VWF by ADAMTS13 dose-dependently under vortex-induced mechanic shear stress (FIG. 7). At concentration of 150 nM, T5-8CUB and T2-8CUB inhibited proteolytic cleavage of VWF by ADAMTS13 by 75% and 100%, respectively (FIGS. 7A and 7B). These data demonstrate the although there may be VWF-binding sites present within the TSP1 repeats and the CUB domains, the cooperative activity among these domains appears to be crucial for productive binding and efficient cleavage of VWF under flow.

### Discussion

The present study demonstrates that multimeric VWF can be readily cleaved by recombinant or plasma ADAMTS13 within 3 min under mechanic-induced shear stress on a mini-vortexer. The cleavage is specific at the Tyr¹⁶⁰⁵-Met¹⁶⁰⁶ bond as shown by the presence of dimers of 176 kDa. The VWF proteolysis is rotation-speed (FIG. 2A) and the ADAMTS13-concentration dependent (FIG. 2B). Addition of EDTA (10 mM) or omission of ADAMTS13 enzyme into the reaction abrogates cleavage of VWF (FIG. 2C), confirming the specific cleavage of VWF by ADAMTS13, not simply by the mechanic-induced shear stress. VWF can also be cleaved by normal human plasma, but not by TTP-patient plasma in presence of heparin (FIG. 2B), indicating that the simple flow based-assay is applicable to determine plasma ADAMTS13 activity in patients with congenital and acquired TTP.

ADAMTS13 does not bind or cleave native VWF in absence of flow shear stress or denaturing regents. An early study has shown that 1,500 s⁻¹ shear rate may be required to detect VWF proteolysis by plasma ADAMTS13 enzyme³⁵. Yet, in a mouse model, thrombi are formed in the venules of the mesentery (shear rate of ˜200-250 s⁻¹) in adamts13^{-/-} mice after topical fusion of calcium ionophore A23187, but not in adamts13+/+ mice or in adamts13^{-/-} mice supplemented with recombinant ADAMTS13 protein via tail vein injection³⁶, indicating that ADAMTS13 and VWF interaction may occur at low shear stress. Consistent with this premise, the data show that the cleavage of VWF is detectable at low vortexing-rotation speed (˜640 rpm); the cleavage product accumulates in a rotation speed-dependent manner, and reaches the plateau at the rotation rates between ˜2,500 rpm and ˜3,200 rpm (with an estimated shear rate of ˜12,000 s⁻¹) (FIG. 2A). On a BIAcore system, the multimeric plasma VWF binds ADAMTS13 at injection rate of 10 μl/min (shear rates ˜500 s⁻¹), but the affinity is not enhanced with increasing injection rates up to 100 μl/min (with shear rate of ˜5,000 S⁻¹) (FIG. 3A). These data indicate that ADAMTS13 may be physiologically important in preventing thrombus formation in both arterioles and venules.

Although the N-terminal domains of ADAMTS13 appear to be sufficient to bind and cleave VWF under denatured and static condition^{22-24;29,} the C-terminal domains are clearly required for recognition of VWF under flow. A removal of the CUB domains (delCUB) or more (MDTCS) abrogates its ability to cleave VWF under vortex-induced mechanic shear stress (FIG. 2C). Yet, these C-terminal truncated variant are able to cleave guanidine-HCl denatured VWF (FIG. 2D) or GST-VWF73 (FIGS. 2E and 2F) or FRETS-VWF73 with more or similar efficacy, compared to full-length ADAMTS13. Analysis on the BIAcore system has also shown that full-length ADAMTS13 binds VWF in high affinity (K_{D} of ˜50 nM). The removal of the CUB domains results in ˜5-fold decrease in the binding affinity (Table 1 and FIG. 3), and further removal of the TSP1 2-8 repeats almost completely abolishes its ability to bind VWF under flow. Again, pre-denatured VWF is able to bind ADAMTS13 substantially, with a K_{D} of ˜330 nM, comparable to that of the construct delCUB (Table 1). These data indicate that the C-terminal TSP1 repeats and CUB domains participating in substrate recognition under flow and the pre-denaturization of VWF exposes additional cryptic sites, which are otherwise not available under flow alone.

To determine whether the CUB domains are sufficient to bind VWF under flow or whether the other adjacent structure is required for binding, the direct binding and competition inhibition assays were performed with various purified C-terminal fragments of ADAMTS13. It was shown that the isolated CUB (CUB) domains are not able to bind VWF under flow detectably on BIAcore system (FIG. 6A) or microtiter plate. Neither does the CUB fragment, nor does the TSP1 2-8 or T5-8 fragment inhibit the cleavage of VWF by recombinant ADAMTS13 in a flow-based assay (FIG. 7). However, addition of the TSP1 5-8 repeats to the CUB domains (construct T5-8CUB) restores the binding affinity toward VWF under flow (K_{D} of ˜212) (FIG. 6) and their inhibitory potency toward cleavage of VWF by ADAMTS13 (FIG. 7). Further addition of the TSP1 2-4 repeats (construct T2-8CUB) increases the affinity by ˜1.5-fold (FIG. 6) and their inhibitory potency (FIG. 7), indicating that the cooperative activity between the distal TSP1 repeats and the CUB domains is critical for productive recognition of VWF under flow.

The data indicate that coagulation factor VIII is a cofactor, as described above, which enhances proteolytic cleavage of VWF by ADAMTS13 by at least 10 fold. These findings for the first time provide the link between the coagulation system and ADAMTS13 metalloprotease, indicating a possible compensatory mechanism for hemophilia A patients and necessary modifications of therapeutic strategies for TTP and hemophilia patient.

In summary, it has been demonstrated that multimeric VWF can be readily cleaved by full-length recombinant and plasma ADAMTS13, but not by the C-terminal truncated variants under the vortex-induced mechanic shear stress. The interaction between VWF and ADAMTS13 under flow is a high affinity one. Although there may be VWF-binding sites in the TSP1 repeats and the CUB domains of ADAMTS13, the cooperative activity between these domains appears to be crucial for productive recognition and cleavage of VWF under flow.

### REFERENCES FOR EXAMPLE I

1. Tsai H M. Physiologic cleavage of von Willebrand factor by a plasma protease is dependent on its conformation and requires calcium ion. Blood 1996; 87:4235-44.
2. Levy G G, Nichols W C, Lian E C et al. Mutations in a member of the ADAMTS gene family cause thrombotic thrombocytopenic purpura. Nature 2001; 413:488-94.
3. Kokame K, Matsumoto M, Soejima K et al. Mutations and common polymorphisms in ADAMTS13 gene responsible for von Willebrand factor-cleaving protease activity. Proc Natl Acad Sci USA 2002; 99:11902-7.
4. Antoine G, Zimmermann K, Plaimauer B et al. ADAMTS13 gene defects in two brothers with constitutional thrombotic thrombocytopenic purpura and normalization of von Willebrand factor-cleaving protease activity by recombinant human ADAMTS13. Br J Haematol 2003; 120:821-4.
5. Assink K, Schiphorst R, Allford S et al. Mutation analysis and clinical implications of von Willebrand factor-cleaving protease deficiency. Kidney Int 2003; 63:1995-9.
6. Savasan S, Lee S K, Ginsburg D, Tsai H M. ADAMTS13 gene mutation in congenital thrombotic thrombocytopenic purpura with previously reported normal VWF cleaving protease activity. Blood 2003; 101:4449-51.
7. Schneppenheim R, Budde U, Oyen F et al. von Willebrand factor cleaving protease and ADAMTS13 mutations in childhood TTP. Blood 2003; 101:1845-50.
8. Kokame K, Miyata T. Genetic defects leading to hereditary thrombotic thrombocytopenic purpura. Semin Hematol 2004; 41:34-40.
9. Pimanda J E, Maekawa A, Wind T et al. Congenital thrombotic thrombocytopenic purpura in association with a mutation in the second CUB domain of ADAMTS13. Blood 2004; 103:627-9.
10. Tsai H M, Lian E C. Antibodies to von Willebrand factor-cleaving protease in acute thrombotic thrombocytopenic purpura. N Engl J Med 1998; 339:1585-94.
11. Zheng X L, Richard K M, Goodnough L T, Sadler J E. Effect of plasma exchange on plasma ADAMTS13 metalloprotease activity, inhibitor level, and clinical outcome in patients with idiopathic and non-idiopathic thrombotic thrombocytopenic purpura. Blood 2004; 103:4043-4049.
12. Niiya M, Uemura M, X. W. Z et al. Increased ADAMTS13 proteolytic activity in rat hepatic stellate cells upon activation in vitro and in vivo. J. Thromb. Haemost. 2006; 4:1063-70.
13. Uemura M, Tatsumi K, Matsumoto M et al. Localization of ADAMTS13 to the stellate cells of human liver. Blood 2005; 106:922-4.
14. Zhou W, Inada M, Lee T P et al. ADAMTS13 is expressed in hepatic stellate cells. Lab Invest 2005; 85:780-8.
15. Shang D, Zheng X W, Niiya M, Zheng X L. Apical sorting of ADAMTS13 in vascular endothelial cells and Madin-Darby canine kidney cells depends on the CUB domains and their association with lipid rafts. Blood 2006; 108:2207-2215.
16. Turner N, Nolasco L, Tao Z, Dong J F, Moake J. Human endothelial cells synthesize and release ADAMTS-13. J Thromb Haemost 2006; 4:1396-1404.
17. Liu L, Choi H, Bernardo A et al. Platelet-derived VWF-cleaving metalloprotease ADAMTS-13. J Thromb Haemost 2005; 3:2536-44.
18. Suzuki M, Murata M, Matsubara Y et al. Detection of von Willebrand factor-cleaving protease (ADAMTS-13) in human platelets. Biochem Biophys Res Commun 2004; 313:212-6.
19. Ono T, Mimuro J, Madoiwa S et al. Severe secondary deficiency of von Willebrand factor-cleaving protease (ADAMTS13) in patients with sepsis-induced disseminated intravascular coagulation: its correlation with development of renal failure. Blood 2006; 107:528-34.
20. Shelat S G, Smith A G, Ai J, X. Z. Inhibitory autoantibodies against ADAMTS-13 in patients with thrombotic thrombocytopenic purpura bind ADAMTS-13 protease and may accelerate its clearance in vivo. J. Thromb.Haemost. 2006; 4:1707-1717.
21. Zheng X, Chung D, Takayama T K et al. Structure of von Willebrand factor-cleaving protease (ADAMTS13), a metalloprotease involved in thrombotic thrombocytopenic purpura. J Biol Chem 2001;276:41059-63.
22. Ai J, Smith P, Wang S, Zhang P, Zheng X L. The Proximal Carboxyl-terminal Domains of ADAMTS13 Determine Substrate Specificity and Are All Required for Cleavage of von Willebrand Factor. J Biol Chem 2005;280:29428-34.
23. Soejima K, Matsumoto M, Kokame K et al. ADAMTS-13 cysteine-rich/spacer domains are functionally essential for von Willebrand factor cleavage. Blood 2003; 102:3232-7.
24. Zheng X, Nishio K, Majerus E M, Sadler J E. Cleavage of von Willebrand factor requires the spacer domain of the metalloprotease ADAMTS13. J Biol Chem 2003;278:30136-41.
25. Wu J J, Fujikawa K, McMullen B A, Chung D W. Characterization of a core binding site for ADAMTS-13 in the A2 domain of von Willebrand factor. Proc. Natl. Acad. Sci. U.S.A 2006; 103:18470-18474.
26. Gao W, Anderson P J, Majerus E M, Tuley E A, Sadler J E. Exosite interactions contribute to tension-induced cleavage of von Willebrand factor by the antithrombotic ADAMTS13 metalloprotease. Proc. Natl. Acad. Sci. U.S.A 2006; 103:19099-19104.
27. Tao Z, Wang Y, Choi H et al. Cleavage of ultralarge multimers of von Willebrand factor by C-terminal-truncated mutants of ADAMTS-13 under flow. Blood 2005; 106:141-3.
28. Tao Z, Peng Y, Nolasco L et al. Role of the CUB-1 domain in docking ADAMTS-13 to unusually large Von Willebrand factor in flowing blood. Blood 2005
29. Majerus E M, Anderson P J, Sadler J E. Binding of ADAMTS13 to von Willebrand factor. J Biol Chem 2005;280:71773-8.
30. Anderson P J, Kokame K, Sadler J E. Zinc and calcium ions cooperatively modulate ADAMTS13 activity. J Biol Chem 2006; 281:850-7.
31. Girma J P, Chopek M W, Titani K, Davie E W. Limited proteolysis of human von Willebrand factor by Staphylococcus aureus V-8 protease: isolation and partial characterization of a platelet-binding domain. Biochemistry 1986; 25:3156-3163.
32. Ashida N, Takechi H, Kita T, Arai H. Vortex-mediated mechanical stress induces integrin-dependent cell adhesion mediated by inositol 1,4,5-trisphosphate-sensitive Ca2+ release in THP-1 cells. J. Biol. Chem. 2003; 278:9327-9331.
33. Ku D N, Glagov S, Moore J E, Jr., Zarins C K. Flow patterns in the abdominal aorta under simulated postprandial and exercise conditions: an experimental study. J. Vasc. Surg. 1989; 9:309-316.

### Example II

### Co-Factor Activity of Coagulation Factor Viii in Cleavage by VWF by ADAMTS13 Metalloprotease

Proteolytic processing of von Willebrand factor (VWF) by ADAMTS13 metalloproteinase is crucial for normal hemostasis. In vitro, cleavage of VWF by ADAMTS13 is slow even at high shear stress and is typically studied in the presence of denaturants. It has now been shown that under shear stress and at physiological pH and ionic strength, coagulation factor VIII (FVIII) accelerates, by a factor of 10, the rate of specific cleavage at the Tyr1605-Met1606 bond in VWF. Multimer analysis reveals that FVIII preferentially accelerates the cleavage of high-molecular-weight multimers. This rate enhancement is not observed with VWF predenatured with 1.5 M guanidine. The ability of FVIII to enhance VWF cleavage by ADAMTS13 is rapidly lost after pretreatment of FVIII with thrombin. A FVIII derivative lacking most of the B domain behaves equivalently to full-length FVIII. In contrast, a derivative lacking both the B domain and the acidic region a3 that contributes to the high-affinity interaction of FVIII with VWF exhibits a greatly reduced ability to enhance VWF cleavage. The data indicate that FVIII plays a role in regulating proteolytic processing of VWF by ADAMTS13 under shear stress, which depends on the high-affinity interaction between FVIII and its carrier protein, VWF.

The following methods are provided to facilitate the practice of Example II. They are not intended to limit the invention in any way.

### Preparation of Recombinant and Native Proteins:

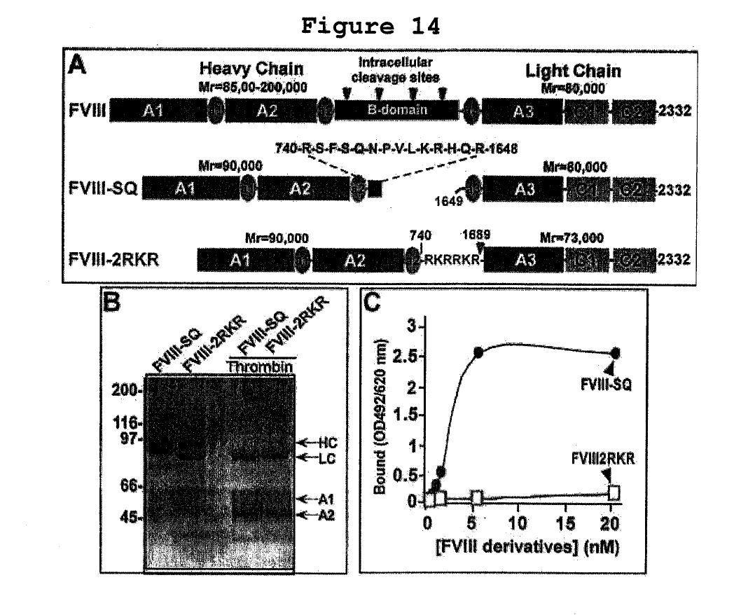

Recombinant human full-length FVIII, obtained as a kind gift from Lisa Regan, Bayer Corporation, was re-purified to remove serum albumin by cation exchange chromatography (22), exchanged into 20 mM HEPES, 0.15 M NaCl, 5 mM CaCl₂, pH 7.5 and stored at −80° C. A B-domainless derivative of FVIII (FVIII-SQ) was constructed using the technique of splicing by overlap extension (23) using human FVIII cDNA (ATCC, Manassas, Va.) as a template. The product was sub-cloned into the pED expression vector obtained as a generous gift from Monique Davis (Wyeth, Cambridge, Mass.) (24). FVIII-SQ lacks residues 744-1637 and has a 14 amino acid linker between the heavy (1-740; A1-A2 domains) and light (1649-2332; a3-A3-C1-C2) chains (FIG. 14A). FVIII-2RKR lacks the entire B domain and acidic region a3 (741-1689). A P ACE/furin recognition site (RKRRKR) was inserted between the heavy (1-740) and the light chains (1690-2332) to facilitate intracellular proteolytic processing (FIG. 14A). Plasmids were transfected into baby hamster kidney (BHK) cells and stable clones were established essentially as described (25). Recombinant FVIII derivatives were purified using procedures described with minor modifications (25). Recombinant vWF was expressed in BHK cells overexpressing PACE/furin and purified from conditioned media by immunoaffinity chromatography using monoclonal antibody RU-8 as described (26). Plasma vWF was purified from cryoprecipitate as described (27). Recombinant ADAMTS13 containing a V5-His tag at the C-terminus was expressed in HEK293 cells and purified according to published procedures (18). Thrombin was prepared from prothrombin and purified as described (28). Protein purity was assessed by SDS-PAGE under reducing conditions, followed by staining with Coomassie Blue. Protein concentrations were determined using the following molecular weights and extinction coefficients (E₂₈₀, 1 mg/ml): FVIII 264,700, 1.22 calculated from amino acid composition (29); FVIII-SQ and FVIII-2RKR 160,000, 1.6 (30); ADAMTS13 195,000,0.68 (18), vWF 250,000, 1.0 (6).

### Cleavage of vWF by ADAMTS13 Under Shear Stress:

Purified plasma or recombinant vWF (37.5 μg/ml or 150 nM) were incubated at 25° C. for 3 min or the indicated times with 50 nM recombinant ADAMTS13 in the absence or presence of FVIII, FVIII-SQ, FVIII-2RKR or FVIIIa (0-40 nM) in 20 mM HEPES, 0.15 M NaCl, 5 mM CaCl₂, 0.5 mg/ml BSA pH 7.5 under constant vortexing at 2,500 rpm. Experiments were performed in 0.2 ml thin-walled PCR tubes (Fisher Scientific, Hampton, N.H.) with a final reaction volume of 20 μl as described previously (18). The reaction was quenched at various times by adding an equal volume of 125 mM Tris, 10% (v/v) glycerol, 2% (w/v) SDS, 0.01% (w/v) bromophenol blue pH 6.8, followed by heating at 100° C. for 5 min. Samples were run on a 5% Tris-glycine SDS-PAGE gel and then transferred to nitrocellulose. The membrane was blocked with 1% (w/v) casein in 20 mM Tris-HCl, 0.15 M NaCl, 0.05% (v/v) Tween 20 (TBSTc) and then incubated with rabbit anti-vWF IgG (DAKO, Carpinteria, Calif.) in TBSTc for 2 hours or overnight at 25° C. Following washing with TBST, the blot was incubated for 1 hr with IRDye® 800CW labeled goat anti-rabbit IgG (LI-COR Bioscience, Lincoln, Nebr.) in TBSTc. An Odyssey Infrared Imaging System (LI-COR Bioscience) was used to quantify the fluorescent signal of the cleavage product (Mr=350K).

### vWF Multimer Analysis:

Following digestion under various conditions, samples were denatured by heating at 60° C. for 20 min in 70 mM Tris, 2.4% (w/v) SDS, 0.67 M urea, 4 mM EDTA pH 6.5 and fractionated in a gel containing 1.5% (w/v) SeaKem HGT agarose (Cambrex, East Rutherford, N.J.). Protein was transferred onto polyvinylidene fluoride membranes (Millipore) by capillary diffusion. Blots were processed for immunodetection as described above.

### Cleavage of vWF by ADAMTS13 Under Denaturing Conditions:

Purified plasma vWF (3.0 μM) was pre-denatured with 1.5 M guanidine at 37° C. for 2 h. Following a 1:10 dilution, vWF was incubated with 12.5 nM of recombinant ADAMTS13 at 37° C. for 1 hour in the absence or presence of FVIII (0-40 nM) in assay buffer. The 350K cleavage product was detected by western blot analysis as described above.

### Binding of FVIII Derivatives to Solid-Phase vWF:

Wells of a microtiter plate were coated with vWF (10 μg/ml) and blocked with 1% casein in PBS, pH 7.4. FVIII-SQ and FVIII-2RKR (0-20 nM) in PBS with 0.1% casein were incubated with immobilized vWF for 1 hour. After washing with PBS, bound FVIII-SQ or FVIII-2RKR was detected in an ELISA format using a monoclonal anti-FVIII antibody (ESH-8) against the C2 domain of FVIII (kindly provided by Dr. Weidong Xiao) and peroxidase-conjugated goat anti-mouse IgG (DAKO, Carpinteria, Calif.).

### Cleavage of FRETS-vWF73:

ADAMTS13 (12.5 nM) and FVIII (0-40 nM) were preincubated for 5 min at room temperature and FRETS-vWF73 substrate (2 μM) in 5 mM Bis-Tris, 25 mMCaCl₂, 0.005% Tween-20, pH 6.0 was then added (31). The cleavage of FRETS-vWF73 was monitored using λ_{EX}=340 nm and λ_{EM}=450 nm at 30° C. with a Wallac 1420 VICTOR³ fluorescent plate reader (Perkin-Elmer Life sciences, Downers Grove, Ill.) to determine initial rates of cleavage.

### Binding of FVIII to ADAMTS13:

Recombinant ADAMTS13 was coupled to a carboxymethylated dextran plasmon resonance chip (2,000 response units; 2-10 ng/mm²) using methods described previously (18). Casein was immobilized in a similar way in the control channel and both surfaces were blocked using 1 M ethanolamine, pH 8.5. FVIII derivatives (0-40 nM) in 20 mM HEPES, 0.15 M NaCl, 5 mM CaCl₂, 0.005% (v/v) Tween 20, pH 7.5 were passed over the chip at a rate of 20 μl/min for 3 min and sensograms were recorded in a BiaCore2000 instrument. After subtraction of non-specific binding, binding curves were analyzed by fitting the data of maximal response units at equilibrium against the concentrations of FVIII derivatives.

### Results

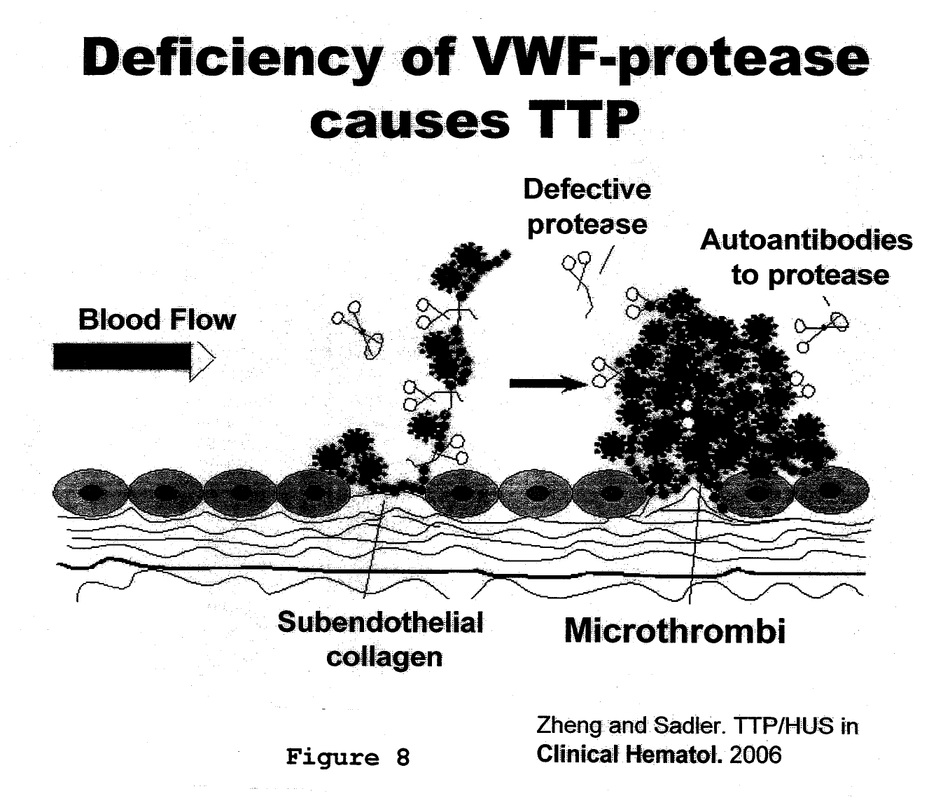

As mentioned above, ADAMTS13 metalloprotease, an enzyme that cleaves an adhesion molecule von Willebrand factor (VWF), is made in liver and secreted into the blood stream. Inability to cleave newly synthesized and released VWF due to congenital or acquired deficiency of ADAMTS13 enzyme leads to an accumulation of VWF in the blood stream, which may then result in an excessive platelet clumping or aggregation, forming widespread blood clots in small arterioles. This disease is referred to as thrombotic thrombocytopenic purpura (TTP). See FIG. 8.

Data presented herein indicate that recombinant ADAMTS13 cleaves VWF less efficiently than the ADAMTS13 in plasma, indicating that there might be cofactors in plasma that are required for enhancement of VWF proteolysis by ADAMTS13. However, exact nature of the cofactors is not known.

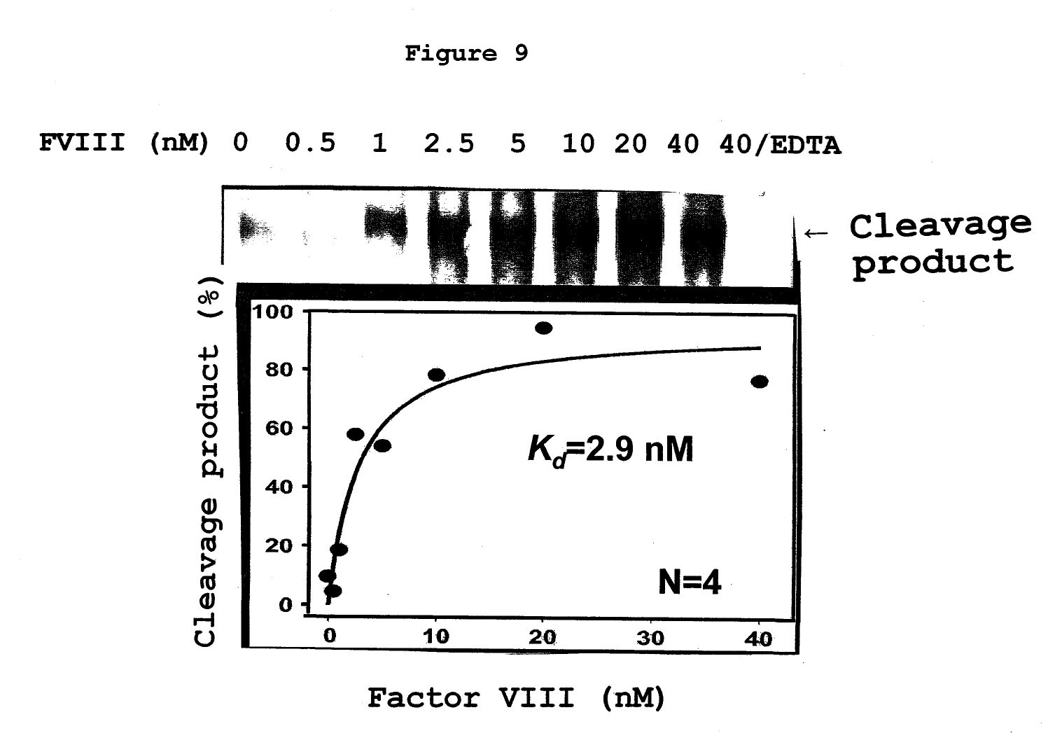

Based on the simple flow-based assay described in the previous example, it has been determined that coagulation factor VIII is one of the cofactors for cleavage of VWF by ADAMTS13. Factor VIII is required for normal hemostasis and blood clotting. Deficiency of factor VIII results in bleeding disorder, namely hemophilia A. Factor VIII is unstable by itself in blood. It almost always binds to VWF for form VWF-FVIII complexes. The question arises whether binding of factor VIII to VWF affects VWF proteolysis by ADAMTS13. It has been shown that recombinant VWF in absence of factor VIII was cleaved relatively slowly. Addition of recombinant factor VIII to the recombinant VWF or plasma-derived VWF significantly enhanced cleavage of VWF by ADAMTS13. See FIG. 9. This enhancement is dose-dependent and time-dependent with the Km of 2-3 nM. This cofactor activity could not be detected when cleavage of VWF was performed under denatured conditions, indicating that conformation change induced by flow allows ADAMTS13 enzyme to bind and cleave VWF-factor VIII complexes.

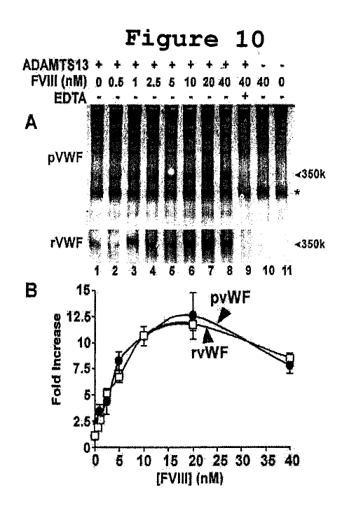

As mentioned previously, FVIII enhances proteolytic cleavage of vWF by ADAMTS13 under shear stress. Purified plasma-derived vWF (37.5 μg/ml or 150 nM) was incubated with recombinant ADAMTS13 (50 nM) for 3 min under constant vortexing in the absence and the presence of various concentrations (0-40 nM) of recombinant FVIII. Proteolysis of vWF was detected by the appearance of an immunoreactive fragment (Mr=350K) representing a disulfide bond linked dimer resulting from cleavage by ADAMTS13 following Tyr1605 in two adjacent subunits within the vWF multimer (1). Cleavage rate and product formation were increased with increasing concentrations of FVIII (FIG. 10). In multiple experiments, ADAMTS13 function, detected in this way, was enhanced by 10-12-fold in the presence of 10-20 nM FVIII (FIG. 10). The concentration of FVIII required for half maximal enhancement in product formation was ˜3.0 nM (FIG. 10), indicating that these increases in product formation occur within the realm of the marginal fractional saturation of vWF monomers within the multimer by FVIII expected in vivo (12).

Enhanced vWF cleavage resulting from buffer artifacts could be excluded because FVIII had been re-purified by cation exchange chromatography, dialyzed and stored in assay buffer lacking BSA. Furthermore, immunoprecipitation with goat anti-FVIII IgG bound to protein G-Sepharose largely eliminated the rate enhancing effects of FVIII added to the cleavage reaction. Other control experiments established the lack of detectable cleavage following the addition of FVIII in the absence of ADAMTS13 (FIG. 10), or addition of EDTA (20 mM) to complete reaction mixtures (FIG. 10). These findings rule out some obvious trivial explanations for the observations.

vWF purified from plasma can contain as much as 1% (w/w) contaminating FVIII (10). The presence of endogenous contaminating FVIII or its proteolytic fragments could obscure the true extent to which added FVIII enhances vWF cleavage. This possibility was assessed using purified recombinant vWF expressed in baby hamster kidney cells and was thus never previously exposed to detectable levels of FVIII. The results with rvWF were equivalent to those obtained using pvWF. With rvWF as substrate, product formation was increased ˜10-fold in the presence of 20 nM FVIII with a half-maximal effect also observed at ˜3 nM (FIG. 10). Any possible endogenous FVIII in vWF purified from plasma is not functional in this assay, possibly owing to its inactivation or degradation during vWF purification.

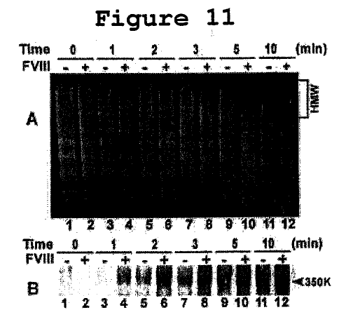

Factor VIII preferentially accelerates cleavage of “high molecular weight” vWF multimers. Detection of the cleaved fragment (350K) facilitates “semi-quantitative” and somewhat defined measurements of ADAMTSI3 function (1). However, it is a potentially misleading measurement because product is only detected following cleavages in two adjacent vWF subunits that are not a requirement for the biologically relevant processing of vWF multimers by ADAMTSI3. The vWF multimer distribution after digestion with ADAMTS13 in the absence and the presence of 20 nM FVIII was assessed. Agarose gel electrophoresis and Western blot analysis revealed a dramatic reduction in high molecular weight multimers ofvWF in the presence of FVIII (FIG. 11A). The degradation of high molecular weight vWF multimers was time-dependent and was also associated with an increase in formation of the degradation product (350K) (FIG. 11B). These findings indicate that the loss of the larger multimers results from proteolytic cleavage of vWF at the Tyr¹⁶⁰⁵-Met¹⁶⁰⁶ bond by ADAMTSI3 and not from nonspecific adsorption or aggregation-related depletion of multimers following their exposure to high shear.

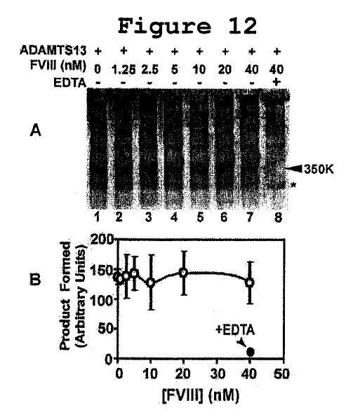

Denaturation of vWF abolishes FVIII effects on ADAMTS13 function. Pre-treatment of vWF with 1.5 M guanidine increases its cleavage by ADAMTS 13 when assessed at low ionic strength (1). To determine whether FVIII affects vWF proteolysis by ADAMTS13 under such conditions that are widely employed to assess enzyme activity, increasing concentrations of FVIII were added to reaction mixtures containing guanidine-denatured vWF (150 nM) and recombinant ADAMTS13 (12.5 nM) in 50 mM HEPES, pH 7.5 and 50 mM NaCl at 37° C. Reaction progress was monitored at various times (0, 5, 10, 30 and 60 min) following initiation by immunodetection of the 350K cleavage product. No increase in cleavage product was detected at any time point in the presence of 20 nM FVIII or in the presence of increasing FVIII concentrations after 1-h incubation (FIG. 12). Thus, FVIII does not play a role in enhancing the digestion of “unfolded” vWF by ADAMTSI3.

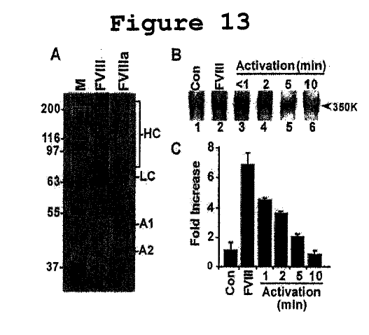

Thrombin activation of FVIII modulates its role in affecting vWF proteolysis. Proteolytic activation of FVIII by thrombin is enhanced when it is bound to vWF (13, 14). The resulting heterotrimeric FVIIIa dissociates from vWF and exhibits labile procoagulant activity because of the rapid dissociation of the A2 subunit (9, 15, 16). FVIII was rapidly activated by the addition of high concentrations of thrombin followed by inhibition of thrombin with hirudin resulting in the quantitative formation of FVIIIa characterized by A1, A2 and A3-C1-C2 fragments (FIG. 13A). At various times following activation, FVIIIa (20 nM) was added to reaction mixtures containing vWF (150 nM) and recombinant ADAMTS13 (50 nM). The 350K cleavage product was detected following a 3 min incubation under constant vortexing. Enhanced product formation rapidly decreased to control levels with a half-life of approximately 2 minutes (FIGS. 13B and 13C). Thus, activation of FVIII by thrombin and the dissociation of FVIIIa from vWF and/or dissociation of the A2 subunit eliminated its ability to enhance cleavage of vWF by ADAMTS13. This points to an additional mechanism that may play a role in regulating ADAMTS13-mediated vWF proteolysis during on-going coagulation.

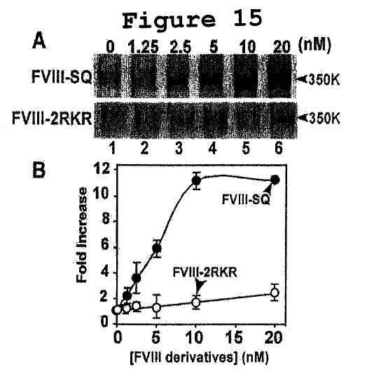

Binding of FVIII to vWF correlates with its ability to enhance vWF cleavage. Two recombinant FVIII derivatives were utilized to investigate the relationship between its ability to bind vWF and the modulation of vWF cleavage by ADAMTSI3. The control construct, the B-domainless FVIII-SQ, contained only 14 residues of the 909 residues in the B-domain (FIG. 14A). The second B-domainless derivative, FVIII-2RKR, was designed with a Pace/Furin site to allow secretion of a two chain species lacking acidic region 3 at the N-terminus of the light chain (FIG. 14A). As expected, SDS-PAGE analysis revealed that the light chain of construct FVIII-2RKR was slightly smaller than that of FVIII-SQ (FIG. 14B). Both FVIII-SQ and FVIII-2RKR are expected to exhibit procoagulant activity, while only FVIII-SQ but not FVIII-2RKR is expected to bind vWF with high affinity (9). Accordingly, the specific activity determined by activated thromboplastin time for FVIII-2RKR (35,000 IV/mg) was roughly comparable to that of FVIII-SQ (10,000±350 IV/mg). FVIII-2RKR bound poorly to immobilized vWF in comparison to FVIII-SQ (FIG. 14C). This finding is in agreement with other studies implicating a role for acidic region 3 in the interaction of FVIII and vWF (13, 17). When assessed in assays for vWF cleavage, FVIII-SQ behaved equivalently to full-length FVIII yielding a ˜10-fold increase in vWF proteolysis by ADAMTS13 (FIG. 15). Half-maximal effects were observed with ˜2.5 nM FVIII-SQ, comparable to the findings with full length FVIII (FIG. 15). These data indicate that most of the central B-domain of FVIII is dispensable for its function in modulating vWF processing. In contrast, FVIII-2RKR failed, even at highest concentration tested, to significantly enhance cleavage of vWF by ADAMTS13 (FIG. 15), indicating that the high affinity binding interaction between FVIII and vWF plays an important role in the ability of FVIII to accelerate ADAMTS13-mediated vWF cleavage.

Factor VIII also interacts with ADAMTS13. Measurements of peptidyl substrate cleavage by ADAMTS13 were employed to assess whether FVIII could directly bind the proteinase and modulate its activity. This approach was pursued because the vWF fragments employed in the peptidyl assay are not expected to bind FVIII. FVIII, FVIII-SQ and FVIII-2RKR increased the initial rate of cleavage of FRETS-vWF73 and GST-vWF73 by a factor of 2 or 3. The data raise the possibility that FVIII and its derivatives may interact with ADAMTS13 and modulate its activity, albeit in a small way. This possibility was further explored by surface plasmon resonance measurements with immobilized full-length ADAMTS13. All three FVIII derivatives bound ADAMTS13 with apparently rapid on rate and off rate. Equilibrium dissociation constants were estimated from the dependence of the plateau signal on the concentration of FVIII derivative injected. Analysis according to the binding of FVIII to equivalent and non-interacting sites with a site concentration well below Kd, yielded equilibrium dissociation constants ranging from 20 nM to 80 nM for the three FVIII derivatives. These affinities are modest in comparison to the concentrations of FVIII (0.3-0.7 nM) and ADAMTS13 (5-7 nM) in plasma. Taken together with the small enhancement in the rate of peptidyl cleavage, the data indicate that direct interactions between FVIII and ADAMTS13, independent of vWF, likely contribute in a minor way to the overall rate enhancing effect on proteolytic cleavage of the macromolecular vWF substrate by ADAMTS13.

The structural elements of FVIII which are required for accelerating proteolytic cleavage of vWF by ADAMTS13 have also been investigated. An isolated light chain of FVIII (FVIII-LC) was sufficient to increase proteolytic cleavage of vWF by ADAMTS13 under fluid shear stress in a concentration-dependent manner. Under mechanically induced fluid shear stress, an FVIII-LC (5 nM) increased the maximal proteolytic cleavage product (˜350 kDa) by 7-8 fold, which was nearly identical to wild type FVIII and B-domain deleted FVIII (FVIII-SQ). The concentration of FVIII-LC achieving the half maximal rate enhancing effect (IC50) was estimated to be ˜2.0 nM. In contrast, an isolated heavy chain of FVIII (FVIII-HC) or a FVIII-LC with an acidic region being deleted (FVIII-LCda3) did not exhibit a significant rate enhancing effect on proteolytic cleavage of vWF by ADAMTS13 under the same conditions. A microtiter binding assay demonstrated that the FVIII-LC bound vWF with a dissociation constant (KD) of ˜17 nM, whereas FVIII-HC and FVIII-LCda3 did not bind the immobilized vWF detectably. These results indicate that binding of FVIII-LC to vWF may be necessary and sufficient to increase the susceptibility of vWF to proteolytic cleavage by ADAMTS13 under fluid shear stress.

To corroborate the biochemical findings, animal studies were performed by investigating plasma vWF antigen levels and multimer distribution using mini-agarose gel electrophoresis and Western blot. Even at baseline, plasma vWF antigen and high molecular weight (HMW) vWF multimers in fVIII knockout mice (fVIII−/−) (n=18) were modestly increased compared with those of wild-type mice (n=14). Upon challenge by a hydrodynamic injection of phosphate-buffered saline (2 ml in 5 seconds), plasma vWF antigen and HMW-vWF multimer were dramatically increased in the fVIII−/− mice (n=10) at 48-hour post-injection. This dramatic increase in plasma vWF antigen and HMW-vWF multimers were abrogated in the fVIII−/− mice with similar hydrodynamic injection of saline containing a plasmid encoding canine FVIII-SQ. At 48-h post injection, plasma canine FVIII-SQ ranged from 150% to 700% of normal canine FVIII activity based on a Coatest assay (n=20). A microtiter binding assay further showed that canine FVIII-SQ bound immobilized murine vWF with a dissociation constant (KD) of ˜1.7 nM. These findings provide further evidence that FVIII is a cofactor that regulates proteolytic processing of vWF by ADAMTS13 under both physiological and pathological conditions. This cofactor activity depends on high affinity interactions between the light chain of FVIII and vWF.

### Discussion

Cofactor proteins play a fundamental role in enhancing proteinase function in the coagulation cascade. The present work was stimulated by the striking similarities in the extreme conditions employed to observe detectable cleavage of vWF by ADAMTS13 and earlier work with coagulation proteinases before the essential contributions of cofactors and membranes were fully appreciated (7).

A search for co-factors that could modulate vWF processing by ADAMTS13 has been hindered by the lack of appropriate assays. The requirement for denaturants such as urea and guanidine and the use of buffers at non-physiological pH and ionic strength could all obscure contributions of other components to proteinase function. The development of a simple shear stress-based assay (18) has provided an opportunity to investigate cofactor-dependent regulation of ADAMTS13 using the native macromolecular substrate and buffer conditions that are more consistent with the physiologic milieu. It has been demonstrated that FVIII accelerates the action of ADAMTS13 on vWF at concentrations that are consistent with the expected marginal saturation ofvWF monomers with FVIII in blood. It is also not surprising that these enhancing effects of FVIII are completely obscured following the use of guanidine to denature the substrate.

The high affinity interaction between FVIII and vWF plays a key role in the ability of FVIII to enhance vWF proteolysis by ADAMTS13 under shear stress. This conclusion follows from the inability of FVIII-2RKR, lacking acidic region 3, to bind with high affinity to vWF or to enhance vWF proteolysis. Alternatively, there is also evidence for the ability of FVIII to bind ADAMTS13 with modest affinity and produce a small increase in catalytic activity. It is clear that this is unlikely to represent the primary mechanism underlying FVIII function in this system particularly because both FVIII and FVIII-2RKR have equivalent effects on the activity of ADAMTS13 toward peptidyl substrates. These observations reflect the features of a three-body problem wherein coupled interactions between FVIII, ADAMTS13 and vWF poise the proteinase on the multimer for enhanced cleavage at Tyr¹⁶⁰⁵. However, it is also possible that the enhancing effects of FVIII reflect its ability to bind vWF and somehow change its conformation and/or its susceptibility to deformation by shear stress.

The observed increase in product formation, resulting from the apparent ability of FVIII to function as a cofactor for vWF cleavage, pales in comparison to the increases in rate associated with cofactor function in the blood coagulation reactions (7). It appears that the 10-fold increase in rate, afforded by FVIII, is sufficient to play an important role in vWF multimer processing in blood where FVIII is constitutively bound to vWF and ADAMTS13 circulates as an active proteinase. However, it is more likely that the true magnitude of the rate enhancing effect is obscured by the complexity of the measurement that relies on immunodetection, with obvious associated problems, of a 350K cleavage product produced only upon cleavage in two adjacent subunits within the multimer. Measurements that rely on the formation of product produced in this way are more than likely to greatly underestimate the rate of the individual cleavage events and the rate at which vWF multimer size is reduced by ADAMTS13.

The effects of FVIII appear far more dramatic when assessed by multimer distributions where the presence of FVIII leads to a selective enhancement in consumption of the larger multimers of vWF. This effect, observed in the absence of denaturants, and rationalized on the basis of the very minimal fractional saturation of vWF with FVIII, provides a potentially cogent explanation for the selective cleavage of “unusually-large” vWF multimers by ADAMTS13 in vivo. This explanation predicts impaired multimer processing in patients with severe hemophilia A (grossly deficient in FVIII) or excessive proteolysis in patients receiving high doses of FVIII.

It has previously been reported that vWF antigen and ristocetin cofactor activity are elevated (−2-fold) in severe hemophilia A patients compared to healthy controls (19). In addition, acquired von Willebrand disease has been reported in a patient receiving prolonged infusion of high dose of recombinant FVIII after surgery (20), although other causes of vWF depletion can not be ruled out. There are seemingly no reports documenting a disproportionate increase in large vWF multimers in severe hemophilia A patients. The reasons for this may include: 1) lack of quantitative methods to document subtle changes in multimer distribution in plasma; 2) difficulties in establishing such a relationship without carefully controlled work because of variability in the multimer patterns between individuals; 3) selective consumption of larger multimers in plasma; or 4) the fact that 10% ADAMTS13 activity is sufficient to proteolytically process unusually large vWF as seen in patients receiving plasma for the treatment of ADAMTS13 deficiency (21). Some of these points may result in the compensation of the bleeding tendency in severe hemophilia A and offer a potential explanation for the heterogeneous bleeding tendency in these patients.

In summary, FVIII functions as a cofactor in accelerating processing of vWF by ADAMTS13 under shear stress. This rate enhancing effect is dependent on the ability of FVIII to bind to vWF with high affinity. The selective action of ADAMTS13 on larger vWF multimers likely arises from the probability of encountering more FVIII molecules bound to the larger multimeric species at physiological concentrations of FVIII and vWF.

### REFERENCES FOR EXAMPLE II

1. Tsai, H M (1996) Physiologic cleavage of von Willebrand factor by a plasma protease is dependent on its conformation and requires calcium ion. Blood 87: 4235-44.
2. Moake, J L, Rudy, C K, Troll, J H, Weinstein, M J, Colannino, N M, Azocar, J, Seder, R H, Hong, SL, Deykin, D (1982) Unusually large plasma factor VIII:von Willebrand factor multimers in chronic relapsing thrombotic thrombocytopenic purpura. N Engl J Med 307: 1432-5.
3. Shim, K, Anderson, P J, Tuley, E A, Wiswall, E, Sadler, J E (2007) Platelet-VWF complexes are preferred substrates of ADAMTS13 under fluid shear stress. Blood 111: 651-657.
4. Furlan, M, Robles, R, Lammle, B (1996) Partial purification and characterization of a protease from human plasma cleaving von Willebrand factor to fragments produced by in vivo proteolysis. Blood 87: 4223-34.
5. Tsai, H M, Lian, E C (1998) Antibodies to von Willebrand factor-cleaving protease in acute thrombotic thrombocytopenic purpura. N Engl J Med 339: 1585-94.
6. Anderson, P J, Kokame, K, Sadler, J E (2006) Zinc and calcium ions cooperatively modulate ADAMTS13 activity. J Bioi Chem 281: 850-7.
7. Mann, K G, Jenny, R J, Krishnaswamy, S (1988) Cofactor proteins in the assembly and expression of blood clotting enzyme complexes. Annu Rev Biochern 57: 915-956.
8. Nishio, K, Anderson, P J, Zheng, X L, Sadler, J E (2004) Binding of platelet glycoprotein Ibalpha to von Willebrand factor domain Al stimulates the cleavage of the adjacent domain A2 by ADAMTS13. Proc Natl Acad Sci USA 101: 10578-83.
9. Lenting, P J, Van Mourik, J A, Mertens, K (1998) The life cycle of coagulation factor VIII in view of its structure and function. Blood 92: 3983-3996.
10. Federici, A B (2003) The factor VIII/von Willebrand factor complex: basic and clinical issues. Haernatologica 88: 3-12.
11. Vlot, A J, Koppelman, S J, van den Berg, M H, Bouma, B N, Sixma, J J (1995) The affinity and stoichiometry of binding of human factor VIII to von Willebrand factor. Blood 85: 3150-3157.
12. Lollar, P, Parker, C G (1987) Stoichiometry of the porcine factor VIII-von Willebrand factor association. J Bioi Chern 262: 17572-17576.
13. Lollar, P, Hill-Eubanks, D C, Parker, C G (1988) Association of the factor VIII light chain with von Willebrand factor. J Bioi Chern 263: 10451-10455.
14. Hill-Eubanks, D C, Lollar, P (1990) von Willebrand factor is a cofactor for thrombincatalyzed cleavage of the factor VIII light chain. J Bioi Chem 265: 17854-17858.
15. Lollar, P, Knutson, G J, Fass, D N (1985) Activation of porcine factor VIII:C by thrombin and factor Xa. Biochemistry 24: 8056-8064.
16. Fay, P J, Smudzin, T M (1992) Characterization of the interaction between the A2 subunit and AIIA3-C1-C2 dimer in human factor VIIIa. J Bioi Chem 267: 1324613250.
17. van den Biggellaar, M, Bierings, R, Storm, G, Voorberg, J, Mertens, K (2007) Requirements for cellular co-trafficking of factor VIII and von Willebrand factor to Weibel-Palade bodies. J Thromb Haemost 5: 2235-2243.
18. Zhang, P, Pan, W, Rux, A H, Sachais, B S, Zheng, X L (2007) The cooperative activity between the carboxyl-terminal TSP-1 repeats and the CUB domains of ADAMTS13 is crucial for recognition of von Willebrand factor under flow. Blood 110: 1887-1894.
19. Grunewald, M, Siegemund, A, Grunewald, A, Konegen, A, Koksch, M, Griesshammer, M (2002) Absence of compensatory platelet activation in patients with severe haemophilia, but evidence for a platelet collagen-activation defect. Platelets 13: 451-458.
20. Rock, G, Adamkiewicz, T, Blanchette, V, Poon, A, Sparling, C (1996) Acquired von Willebrand factor deficiency during high-dose infusion of recombinant factor VIII. Br J Haematol 93: 684-687.
21. Furlan, M, Lammle, B (1998) Deficiency of von Willebrand factor-cleaving protease in familial and acquired thrombotic thrombocytopenic purpura. Baillieres Clin Haematol 11: 509-14.
22. Fay, P J, Haidaris, P J, Smudzin, T M (1991) Human factor VIIIa subunit structure. Reconstruction of factor VIIIa from the isolated AIIA3-C1-C2 dimer and A2 subunit. J Bioi Chem 266: 8957-8962.
23. Horton, R M, Hunt, H D, Ho, S N, Pullen, J K, Pease, L R (1989) Engineering hybrid genes without the use of restriction enzymes: gene splicing by overlap extension. Gene 77: 61-68.
24. Kaufman, R J, Davies, M V, Wasley, L C, Michnick, D (1991) Improved vectors for stable expression of foreign genes in mammalian cells by use of the untranslated leader sequence from EMC virus. Nucl Acids Res 19: 4485-4490.
25. Toso, R, Camire, R M (2004) Removal of B-domain Sequences from Factor V Rather than Specific Proteolysis Underlies the Mechanism by Which Cofactor Function Is Realized. J Bioi Chem 279: 21643-21650.
26. Lankhof, H, Damas, C, Schiphorst, M E, Ijsseldijk, M J, Bradee, M, Furlan, M, de Groot, PG, Sixma, J J, Vink, T (1999) Recombinant vWF type 2A mutants R834Q and R834W show a defect in mediating platelet adhesion to collagen, independent of enhanced sensitivity to a plasma protease. Thromb Haemost 81: 976-983.
27. Zheng, X L, Nishio, K, Majerus, E M, Sadler, J E (2003) Cleavage of von Willebrand factor requires the spacer domain of the metalloprotease ADAMTS13. J Biol Chem 278: 30136-41.
28. Lundblad, R L, Kingdon, H S, Mann, K G (1976) Thrombin. Methods Enzymol45: 156176.
29. Gill, S C, von Hippel, P H (1989) Calculation of protein extinction coefficients from amino acid sequence data. Anal Biochem 182: 319-326.
30. Curtis, J E, Helgerson, S L, Parker, E T, Lollar, P (1994) Isolation and characterization of thrombin-activated human factor VIII. J Biol Chem 269: 6246-6251.
31. Kokame, K, Nobe, Y, Kokubo, Y, Okayama, A, Miyata, T (2005) FRETS-VWF73, a first fluorogenic substrate for ADAMTS13 assay. Br J Haematol 129: 93-100.

### Example III

### von Willebrand Factor Cleaved from Endothelial Cells by ADAMTS13 Remains Ultralarge in Size

von Willebrand factor (VWF) is synthesized in all vascular endothelial cells and megakaryocytes [1]. The mature VWF is stored in the Weibel-Palade bodies of endothelial cells and the agranules of platelets. A small fraction of VWF is constitutively secreted from the cells. Upon stimulation by thrombin, hormones, and inflammatory cytokines, VWF is released from the Weibel-Palade bodies of the endothelial cells as ultralarge (UL) VWF, which forms polymers and remains attached to the endothelial cell surface via poorly characterized mechanisms, although recent reports have indicated that UL VWF might be anchored on endothelial cells via interactions with P-selectin [2] and avb3 integrin [3].

Proteolytic cleavage of the newly synthesized UL VWF on endothelial cells by ADAMTS13 may be critical for the maintenance of normal hemostasis and the inflammatory response. For instance, certain mutations in the vwf gene may result in increased proteolysis of VWF by ADAMTS13 and significant reduction of VWF multimer sizes, leading to compromised hemostatic function, as seen in patients with von Willebrand disease [4]. Conversely, an inability to cleave the newly released UL VWF strings [5,6], owing to a hereditary or acquired deficiency of ADAMTS13, may result in an accumulation of UL VWF that results in the formation of disseminated thrombi in the microvasculature, as in patients with thrombotic thrombocytopenic purpura (TTP) [7]. Moreover, ADAMTS13 deficiency appears to result in increased leukocyte rolling on unstimulated veins, leukocyte adhesion, and extravasation of neutrophils in the inflamed veins [8]. All of these processes seem to depend on the presence of UL VWF multimers [8]. Therefore, the removal of cell-bound UL VWF by ADAMTS13 may help to attenuate systemic inflammatory responses in addition to arterial thrombosis.

In contrast to proteolytic cleavage of VWF in solution, which requires denaturant (urea [9] or guanidine [10]) in a low ionic strength and alkaline buffer or high shear stress [11,12], the removal of UL VWF polymers on cultured endothelial cells by ADAMTS13 occurs very efficiently under various shear stresses [5,6]. The rapid removal of the UL VWF strings by infused recombinant ADAMTS13 also occurs in both arteries (high shear) and venules (low shear) in Adamts13)/) mice [13]. A recent study has shown that UL VWF strings on histamine-stimulated endothelial cells can be removed by ADAMTS13 in the absence of flow shear stress [14].

The following methods are provided to facilitate the practice of Example III. They are not intended to limit the invention in any way.

### Immunofluorescent Microscopy:

Human umbilical vein endothelial cells (HUVECs) at passages 2 and 3 grown on cover slides were stimulated at 37° C. with histamine (100 lM) in phosphate-buffered saline (PBS) for 2 min. After being washed with PBS, the cells were treated for 2 min at 37° C. without (16Ai, buffer) or with (16Aii) 10 nM recombinant full-length ADAMTS13 (FL-AD13). After being washed with Hepes, the cells were fixed with 4% paraformaldehyde in PBS without permeabilization. The fixed cells were incubated for 2 h with mouse or rabbit anti-VWF IgG (1:100) (Dako), and then with Cy3-labeled IgG (1:50) (Invitrogen) for 30 min. The cell nuclei were stained with 4′,6-diamidino-2-phenylindole in the mounting medium (Vector Laboratories). The images were collected under a Leica inverted fluorescence microscope (Witzlar, Germany) with SPOT advanced software version 4.6.

Histamine-stimulated and washed cells were treated with 10 nM ADAMTS13 variants truncated after the eighth thrombospondin type 1 (TSP1) repeat (16-Di, delCUB), after the spacer domain (16-Dii, MDTCS), after the first TSP1 repeat (16-Diii, MDT), and after the metalloprotease domain (Div, M), as in (16A). The cells were fixed and stained as in (16A). Three independent experiments were performed, and the results were consistent.

### ELISA:

The VWF antigen levels determined by ELISA (16Ci) and the proteolytic cleavage products determined by 5% sodium dodecylsulfate polyacrylamide gel electrophoresis and western blot (ii) in the conditioned media of HUVECs, after being treated for 5 min with buffer alone (buffer) or with recombinant full-length ADAMTS13 (FLA13) and various C-terminal truncated variants, including delCUB,MDTCS,MDT, and M, as described in (16A). Three independent experiments were performed (n=3). The means±SDs are shown in (16Ci). ANOVA was performed to compare the VWF antigen levels among various groups. There was no statistically significant difference in the amount of VWF antigen released from cells treated with MDT or M as compared with the buffer control.

### VWF Multimer Distribution:

Agarose gel (1%) electrophoresis and western blot with anti-VWF IgG were used to determine the VWF multimer distribution in the conditioned media collected from histamine-stimulated and washed HUVECs that were treated with recombinant ADAMTS13 (10 nM) for 0-60 min in the absence of flow shear stress (16Ei, lanes 2-8) and in the presence of flow shear stress (up to 25 dynes cm)₂) (16Eii, lanes 3-7). Normal human plasma (NHP, 0.25 lL) was used as a control. In addition, VWF released from HUVECs immediately after stimulation with histamine (100 lM) was used for other controls (16Ei, lane 9; 16Eii, lane 8). EDTA (20 mM) was added to the ADAMTS13-treated conditioned media to block the potential cleavage of UL VWF in solution. Approximately the same amount of VWF antigen was loaded into each lane (except for lane 2 in both panels, where high concentrations were difficult to achieve).

### Results

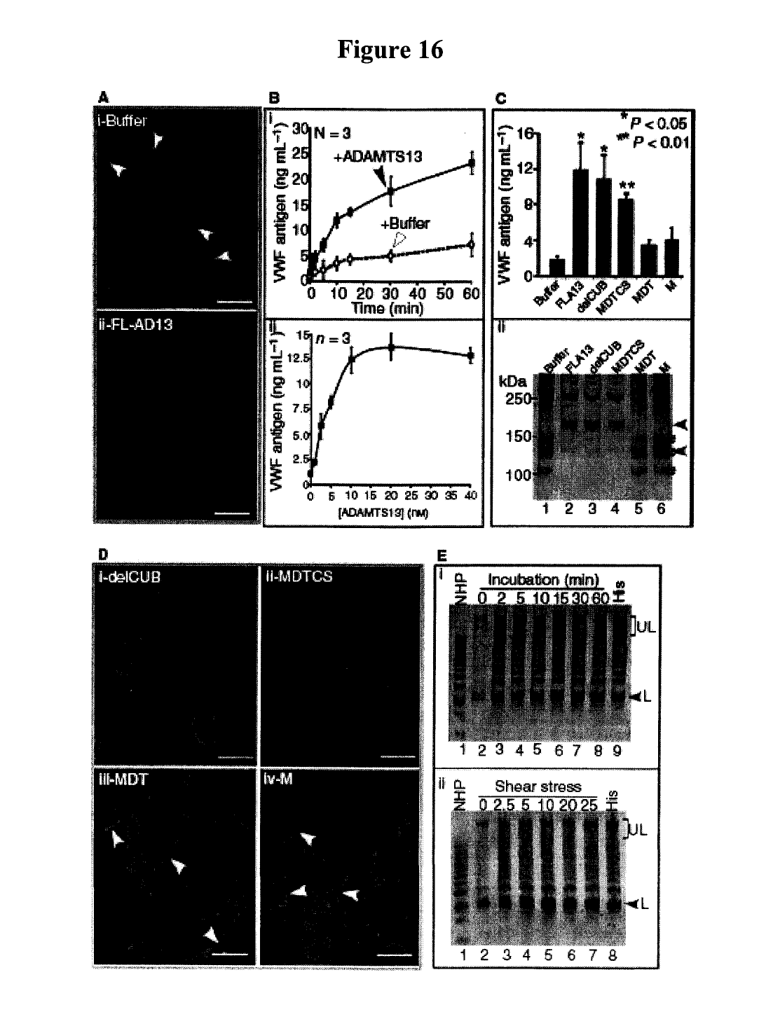

It remains poorly understood what ADAMTS13 domains are required for cleavage of cell-bound UL VWF, and whether proteolytic cleavage of UL VWF by ADAMTS13 on endothelial cell membranes is sufficient to reduce the multimer sizes of UL VWF in the presence or absence of flow shear stress. In this study, immunofluorescence microscopy demonstrated that UL VWF polymers were readily formed on the membranes of human umbilical vein endothelial cells (HUVECs) after stimulation for 2 min with histamine (100 lM) in phosphate-buffered saline (PBS) (FIG. 16Ai). Recombinant ADAMTS13 (10 nM) (FIG. 16A ii) or normal human plasma (1:2 dilution) in an assay buffer (20 mM Hepes, pH 7.4, 150 mM NaCl, 5 mM CaCl₂) efficiently removed all surface-bound UL VWF polymers within 5 min, in the absence of flow shear stress. Plasma (1:2 dilution) from a patient with acquired idiopathic TTP with a known high titer of anti-ADAMTS13 IgG autoantibodies, however, did not result in the removal of the cell-bound UL VWF polymers under the same conditions. These results are consistent with what has been reported by Turner et al. [14], and form the basis for further study of the structural and functional relationships of ADAMTS13 to the cleavage of UL VWF at the cellular level.

To better quantify the amount of UL VWF released from endothelial cells, an enzyme-linked immunosorbent assay, using two polyclonal anti-VWF antibodies, was employed as described previously [15]. In these experiments, HUVECs cultured on six-well plates were stimulated with histamine (100 lM) for 2 min, washed with PBS and then incubated for 0-60 min without or with a fixed concentration (10 nM) of purified recombinant ADAMTS13 in 20 mM Hepes, 150 mM NaCl, and 5 mM CaCl2 (pH 7.4) (FIG. 16Bi), or for 5 min with various concentrations of recombinant ADAMTS13 (0-40 nM) (FIG. 16B ii). The amount of VWF antigen in the conditioned media was increased in an incubation time-dependent and ADAMTS13 concentration-dependent manner (FIG. 16B). After 60 min of incubation with purified recombinant ADAMTS13 (10 nM), the VWF antigen level in the conditioned media (˜24 ng mL)1) was approximately four-fold higher than that in the buffer-treated control (˜6 ng mL)1) (FIG. 16Bi). The concentration of ADAMTS13 that achieved the half-maximal levels of VWF released from endothelial cells was approximately 3.0 nM (FIG. 16B ii), which is within the physiologic range of ADAMTS13 in plasma.

To determine whether or not the C-terminal domains of ADAMTS13 were required for cleavage of cell-bound UL VWF, histamine-stimulated and washed HUVECs were incubated with 10 nM purified ADAMTS13 and various C-terminal truncated variants that have been well characterized previously [12]. The remaining UL VWF on endothelial cells was determined by immunofluorescence microscopy, using polyclonal anti-VWF IgG (Dako). It was found that the variants lacking the CUB domains (delCUB) (FIG. 16Di) and truncated after the spacer domain (MDTCS) (FIG. 16D ii) also efficiently removed UL VWF polymers from histamine-stimulated endothelial cells, similar to full-length ADAMTS13 (FIG. 16A ii). However, the variant truncated after the first thrombospondin type 1 (TSP1) repeat (MDT) (FIG. 16Diii) or the metalloprotease domain (M) (FIG. 16D iv) exhibited markedly impaired activity in removing the cell-bound UL VWF under the same conditions. The removal of cell-bound UL VWF polymers was consistent with the approximately three-fold to four-fold increase in VWF antigen in the conditioned medium after incubation of the histamine-stimulated endothelial cells with full-length ADAMTS13, delCUB, and MDTCS, but not with MDT and M or buffer alone (FIG. 16Ci). The proteolytic cleavage products (176 kDa and 140 kDa) were also detectable in the conditioned media of HUVECs treated with FL-A13, delCUB, and MDTCS, but not with MDT and M or buffer alone (FIG. 16Cii). The amount of VWF antigen detected in the MDTCS-treated medium appeared to be slightly lower than the amounts detected in the FL-A13-treated and delCUB-treated media (FIG. 16C ii), but such a difference was not statistically significant (P>0.05). The reduced amount of cleavage products in the MDTCS-treated medium as compared with full-length ADAMTS13 and delCUB may be a result of imperfect protein loading, rather than the lower proteolytic activity. Thus, the results indicate that the Cys-rich and spacer domains are required for recognition of the cell-bound UL VWF, but that the TSP1 2-8 repeats and the CUB domains are dispensable. These results, however, do not fully agree with those reported elsewhere [14]. The reason for the discrepancy is not completely understood, but may be related to the assay methodologies used. The domain requirement for proteolytic cleavage of cell-bound UL VWF is reminiscent of that seen in the cleavage of soluble VWF by ADAMTS13 under denaturing conditions [16,17], indicating that membrane association of UL VWF results in a conformational change that permits ADAMTS13 binding in the absence of fluid shear stress. The mechanism underlying such a conformational change remains to be determined.

To determine whether proteolytic cleavage of cell-bound UL VWF by ADAMTS13 was sufficient to reduce UL VWF multimer size, the conditioned media were assessed by sodium dodecylsulfate (SDS)-agarose (1%) gel electrophoresis and western blot, as previously described [15]. Unexpectedly, both in the absence of flow (FIG. 16Ei) and in the presence of flow (2.5 dynes cm⁻²) (FIG. 16E ii), VWF multimers released into the conditioned media after being incubated with ADAMTS13 (10 nM) were indistinguishable from those in the conditioned media after being incubated with buffer alone or immediately after histamine stimulation (FIG. 16Ei,ii). When compared with normal human plasma, the conditioned media were highly enriched in ULVWF (FIG. 16Ei,ii). Incubation of the histamine-stimulated endothelial cells with recombinant ADAMTS13 (10 nM) for up to 60 min in the absence of flow (FIG. 16Ei, lanes 2-8) or for 5 min under various flow shear stresses up to ˜25 dynes cm⁻²(FIG. 16Eii, lanes 3-7) did not appear to alter the VWF multimer distribution in the conditioned media, as compared with the UL VWF released from the Weibel-Palade bodies after histamine stimulation (FIG. 16Ei, lane 9; FIG. 16Eii, lane 8), although some of the low molecular weight VWF maybe formed by proteolytic degradation of the UL VWF in the conditioned medium during storage. Addition of 0.1% protease inhibitor cocktail (Sigma) reduced the intensity of the smaller VWF bands. Nevertheless, the VWF cleaved from endothelial cells by ADAMTS13 remains UL. This indicates that further proteolytic processing downstream by ADAMTS13 or other leukocyte proteases [18], which is likely to occur in the small arteries and capillaries, where high fluid shear stress and, perhaps, physiologic cofactors such as factor VIII[19] and platelets[20] are present, may be necessary to eliminate the UL VWF polymers.

### REFERENCES FOR EXAMPLE III

1. Wagner D D, Marder V J. Biosynthesis of von Willebrand protein by human endothelial cells: processing steps and their intracellular localization. J Cell Biol 1984; 99: 2123-30.
2. Padilla A, Moake J L, Bernardo A, Ball C, Wang Y, Arya M, Nolasco L, Turner N, Berndt M C, Anvari B, Lopez J A, Dong J F. P-selectin anchors newly released ultra-large von Willebrand factor multimers to the endothelial cell surface. Blood 2004; 103: 2150-6.
3. Huang J, Roth R, Heuser J E, Sadler J E. Integrin {alpha}v{beta}3 on human endothelial cells binds von Willebrand factor strings under fluid shear stress. Blood 2008; 113: 1589-697.
4. Sadler J E. von Willebrand factor: two sides of a coin. J Thromb Haemost 2005; 3: 1702-9.
5. Dong J F, Moake J L, Nolasco L, Bernardo A, Arceneaux W, Shrimpton C N, Schade A J, McIntire L V, Fujikawa K, Lopez J A. ADAMTS-13 rapidly cleaves newly secreted ultralarge von Willebrand factor multimers on the endothelial surface under flowing conditions. Blood 2002; 100: 4033-9.
6. Dong J F, Moake J L, Bernardo A, Fujikawa K, Ball C, Nolasco L, Lopez J A, Cruz M A. ADAMTS-13 metalloprotease interacts with the endothelial cell-derived ultra-large von Willebrand factor. J Biol Chem 2003; 278: 29633-9.
7. Moake J L. Thrombotic thrombocytopenic purpura: the systemic clumping plague. Annu Rev Med 2002; 53: 75-88.
8. Chauhan A K, Kisucka J, Brill A, Walsh M T, Scheiflinger F, Wagner D D. ADAMTS13: a new link between thrombosis and inflammation. J ExpMed 2008; 205: 2065-74.
9. Furlan M, Robles R, Lammle B. Partial purification and characterization of a protease from human plasma cleaving von Willebrand factor to fragments produced by in vivo proteolysis. Blood 1996; 87: 4223-34.
10. Tsai H M. Physiologic cleavage of von Willebrand factor by a plasma protease is dependent on its conformation and requires calcium ion. Blood 1996; 87: 4235-44.
11. Tsai H M, Sussman I, Nagel R L. Shear stress enhances the proteolysis of von Willebrand factor in normal plasma. Blood 1994; 83: 2171-9.
12. Zhang P, Pan W, Rux A H, Sachais B S, Zheng X L. The cooperative activity between the carboxyl-terminal TSP-1 repeats and the CUB domains of ADAMTS13 is crucial for recognition of von Willebrand factor under flow. Blood 2007; 110: 1887-94.
13. Chauhan A K, Motto D G, Lamb C B, Bergmeier W, Dockal M, Plaimauer B, Scheiflinger F, Ginsburg D, Wagner D D. Systemic antithrombotic effects of ADAMTS13. J Exp Med 2006; 203: 767-76.
14. Turner N, Nolasco L, Dong J F, Moake J. ADAMTS-13 cleaves long von Willebrand factor multimeric strings anchored to endothelial cells in the absence of flow, platelets or conformation-altering chemicals. J Thromb Haemost 2009; 7: 229-32.
15. Niiya M, Endo M, Shang D, Zoltick P W, Muvarak N E, Cao W, Jin S Y, Skipwith C G, Motto D G, Flake A W, Zheng X L. Correction of ADAMTS13 deficiency by in utero gene transfer of lentiviral vector encoding ADAMTS13 genes. Mol Ther 2009; 17: 34-41.
16. Gao W, Anderson P J, Majerus E M, Tuley E A, Sadler J E. Exosite interactions contribute to tension-induced cleavage of von Willebrand factor by the antithrombotic ADAMTS13 metalloprotease. Proc Natl 6 Acad Sci USA 2006; 103: 19099-104.
17. Zheng X L, Nishio K, Majerus E M, Sadler J E. Cleavage of von Willebrand factor requires the spacer domain of the metalloprotease ADAMTS13. J Biol Chem 2003; 278: 30136-41.
18. Raife T J, Cao W, Atkinson B S, Bedell B, Montgomery R R, Lentz S R, Johnson G F, Zheng X L. Leukocyte proteases cleave von Willebrand factor at or near the ADAMTS13 cleavage site. Blood 2009; DOI: 7 10.1182/blood-2009-01-195461.
19. Cao W J, Krishnaswamy S, Camire R M, Lenting P J, Zheng X L. Factor VIII accelerates proteolytic cleavage of von Willebrand factor by ADAMTS13. Proc Natl Acad Sci USA 2008; 105: 7416-21.
20. Shim K, Anderson P J, Tuley E A, Wiswall E, Sadler J E. Platelet-VWF complexes are preferred substrates of ADAMTS13 under fluid shear stress. Blood 2007; 111: 651-7.

While the invention has been described in detail and with reference to specific examples thereof, it will be apparent to one skilled in the art that various changes and modifications can be made therein without departing from the scope thereof.

What is claimed is:

1. A method for analyzing the VWF cleaving action of ADAMTS13 and variants thereof, comprising:
a) providing VWF;
b) contacting said VWF with intact ADAMTS13 and truncated variants thereof under conditions suitable for enzymatic cleavage of VWF;
c) determining the amount of VWF cleavage in the presence of full length ADAMTS13 relative to that observed in the presence of said truncated variants, thereby identifying a minimal ADAMTS13 sequence suitable to effect cleavage of VWF, said method optionally being performed under flow.

2. The method of claim 1, further comprising the addition of a test compound which modulates ADAMTS13 mediated cleavage of VWF.

3. The method of claim 2, wherein said test compound inhibits cleavage of VWF.

4. The method of claim 2, wherein said test compound augments cleavage of VWF.

5. The method of claim 4, wherein said compound is Factor VIII.

6. A method for diagnosing TTP in a patient comprising;
a) obtaining a biological sample comprising VWF and ADAMTS13;
b) subjecting said sample to vortex induced shear stress; and
c) comparing the level of VWF cleavage in said biological sample relative to an identically treated sample from a normal patient, a reduction in VWF cleavage relative to that observed in said normal patient sample being indicative of TTP.

7. The method of claim 6, wherein said sample is selected from the group consisting of blood, serum, and plasma.

8. A method for alleviating the symptoms of a disease in a patient in need thereof, comprising administration of an effective amount of ADAMTS13 and FACTOR VIII in a biologically compatible medium, wherein said disease is selected from the group consisting of TTP, stroke, and myocardial infarction.

9. A method as claimed in claim 8, wherein said ADAMTS13 and said FACTOR VIII are produced recombinantly and purified.

10. The method of claim 9, wherein said proteins are administered systemically.

11. The method of claim 9, wherein said purified proteins are directly infused into a patient, thereby inhibiting or preventing formation of a thrombus.


SEQUENCE LISTING (5 sequences)

SEQ ID NO: 1 - protein, 9 aa, Artificial Sequence
  Synthetic Sequence
EAPDLVLQR

SEQ ID NO: 2 - protein, 5 aa, Artificial Sequence
  Synthetic Sequence
DDDDK

SEQ ID NO: 3 - protein, 30 aa, Artificial Sequence
  Synthetic Sequence
AAQPARRARRTKLALDTKDDDDKHVWTPVA

SEQ ID NO: 4 - protein, 6 aa, Artificial Sequence
  Synthetic Sequence
RKRRKR

SEQ ID NO: 5 - protein, 14 aa, Artificial Sequence
  Synthetic Sequence
RSFSQNPVLKRHQR